(19) United States
(12) Patent Application Publication
(10) Pub. No.: US 2011/0077655 A1
(43) Pub. Date: Mar. 31, 2011

(54) Vertebral Body Spool Device

(76) Inventors: Michael A. Fisher, Grayson, GA (US); Thomas M. DiMauro, Raynham, MA (US); John Riley Hawkins, Raynham, MA (US); Douglas Hester, Raynham, MA (US); Michael J. O'Neil, Raynham, MA (US); Jamie Lockwood, Raynham, MA (US); Matthew Parsons, Raynham, MA (US)

(21) Appl. No.: 12/889,385

(22) Filed: Sep. 23, 2010

Related U.S. Application Data
(60) Provisional application No. 61/246,051, filed on Sep. 25, 2009.
(60) Provisional application No. 61/256,102, filed on Oct. 29, 2009.

(51) Int. Cl. A61B 17/60

(52) U.S. Cl. 606/90

(57) ABSTRACT

A mandrel is inserted into the tissue to be distracted and a filament is repeatedly wrapped around the mandrel to make a spool. As the mandrel turns, the filament coils around the mandrel to create of spool of increasing diameter. This spool can be used as a distractor, tamp, or implant. A spool implant can be cemented to further stabilize the device in situ, such as a vertebral body. The filament deposition upon the mandrel is controlled by rotation and the volume creation is incremental. This makes the spool ideal for creating highly controlled distraction forces or insertion of bulking material. In some embodiments, fibers having a low glass transition temperature are used.

### CONTINUING DATA

This non-provisional patent application claims priority from co-pending U.S. Provisional Application Ser. No. 61/246,051, filed Sep. 25, 2009, entitled “Vertebral Body Spool Device” (DEP6257USPSP1) and co-pending U.S. Provisional Application Ser. No. 61/256,102, filed Oct. 29, 2009, entitled “Vertebral Body Spool Device.” (DEP6257USPSP2), the specifications of which are incorporated by reference in their entireties herein.

### BACKGROUND OF THE INVENTION

In vertebroplasty, the surgeon seeks to treat a compression fracture of a vertebral body by injecting bone cement such as PMMA into the fracture site. In one clinical report, Jensen et al., AJNR: 18 Nov. 1997, Jensen describes mixing two PMMA precursor components (one powder and one liquid) in a dish to produce a viscous bone cement; filling 10 ml syringes with this cement, injecting it into smaller 1 ml syringes, and finally delivering the mixture into the desired area of the vertebral body through needles attached to the smaller syringes.

Vertebral body augmentation requires the creation of a space to receive a bulking agent. Preferably, the space creating technology/technique will restore any lost height of the vertebral body. In one such technique, U.S. Pat. No. 5,108,404 (“Scholten”) discloses inserting an inflatable device within a passage within the vertebral body, inflating the balloon to compact the cancellous bone and create an enlarged void, and finally injecting bone cement into the void.

US Patent Publication 2004-0249464 (Bindseil) discloses an implant for use in fusing adjacent bony structures. The implant comprises a plurality of pieces of bone and a flexible mechanism including one or more flexible, elongate, biocompatible connectors interconnecting the pieces of bone.

US Patent Publication 2007-0123986 (Schaller) discloses methods of separating, supporting or both separating and supporting layers of tissue in the human spine. Such methods generally comprise inserting at least one member between layers of tissue in the human spine and changing the configuration of the member to define a support structure between the tissue layers. Schaller also describes Insertable Flexible Links therein.

US Patent Publication 2007-0149978 (Shezif) discloses a device (10) for distracting and supporting two substantially opposing tissue surfaces in a patient's body, to be introduced within the tissue surfaces in a minimally invasive procedure. The device comprises: a wrapping element (12); and an expandable structure (24) insertable between the two substantially opposing support surfaces of the wrapping element, adapted to be expanded between the two substantially opposing surfaces to a predetermined dimension. See e.g., FIGS. 6a-6d.

PCT Patent Publication WO2006/072941 (Siegal) discloses a device for introduction into a body in a straight configuration and assuming within the body a predefined curved configuration, includes an elongated element formed from a number of segments interconnected so as to form effective hinges therebetween. When the elongated element is confined to a straight state, the effective hinges transfer compressive forces from each segment to the next so that the elongated element can be pushed to advance it through a conduit. When the elongated element is not confined to a straight state, the effective hinges allow deflection of each segment relative to adjacent segments until abutment surfaces of the segments come into abutment, thereby defining a fully flexed state of the elongated element with a predefined curved configuration. The device can be produced with a wide range of two-dimensional and three-dimensional curved forms, and has both medical and non-medical applications.

PCT Patent Publication WO2009006432 (Synthes) discloses a flexible chain implant for insertion into an interior volume of a vertebral body. The implant may be implanted in an insertion position for sliding through a cannula and is flexible for packing into the interior volume in an implanted configuration. The implant randomly separates in the implanted configuration. The implant includes a top member and a bottom member, wherein the top and bottom members are coupled to one another at a coupled portion. The top and bottom members preferably each include an inner surface such that the inner surfaces include a plurality of alternating projections and recesses so that the projections are received within the recesses in an insertion position. Alternatively, the implant may include a plurality of substantially non-flexible bodies and a plurality of substantially flexible links interconnecting the bodies. The non-flexible bodies include a plurality of facets and/or abutment surfaces.

Review of the literature indicates that conventional vertebral body augmentation art does not contemplate a spool of material as a tamp or an implant, except as it is wound perpendicular to the pedicular trajectory. Moreover, the interbody spacer art does not contemplate a spool of material as an implant or distractor. Likewise, the hard tissue bulking art does not describe a spool of material as a tamp or implant, and the soft tissue bulking art does not describe a spool of material as a distracter or implant. Lastly, the method of creating a hard tissue defect or volume using a spool has not been described

### SUMMARY OF THE INVENTION

The present invention addresses the need to create a volume of material or distract a volume in a tissue in a highly controlled, minimally invasive manner.

In accordance with the present invention, a thin mandrel is inserted into the tissue to be distracted and a filament is repeatedly wrapped around the mandrel to make a spool. Preferably, the mandrel is spun about the axis of its insertion trajectory in order to accomplish the wrapping of the filament thereon. As the mandrel turns, the filament wraps around the mandrel to create a spool of increasing diameter. This in situ-created spool can then be used as a distractor, tamp, or implant. In some embodiments, a spool implant can be further cemented or sintered to further stabilize the device in situ. The filament's deposition upon the mandrel is controlled by the clinician's controlling the rotation of the mandrel. Because the volume creation achievable through this procedure is incremental, the spool is highly suitable for creating highly controlled distraction forces or insertion of bulking material.

In preferred embodiments, the spool is used for distraction of a vertebral body compression fracture. In particular, a longitudinal rod having a distal mandrel is inserted into the vertebral body tissue space and spun around the rod axis so that filament material is wound tightly onto the mandrel. As the winding grows in diameter, it fills the vertebral body, displacing marrow, bone, fibrous tissues, and sinuses. Because each individual turn of the filament adds a controlled thickness to the spool, the resistance to winding can be tailored through mandrel and filament design and/or insertion instrument design. Winding the filament around the mandrel creates a spool assembly of growing diameter that acts as a bone tamp. If the mandrel/filament spool assembly is left in situ, the assembly becomes a vertebral body augmentation implant. If the winding process is reversed, the filament and mandrel can be removed in a minimally invasive manner, thereby leaving a bone void that can be filled with a bone cement. In some embodiments, multiple assemblies (such as bilateral assemblies) can be created in situ, as needed. Some assemblies can be used as tamps while others act as implants. The material and functional characteristics of the components of the invention (e.g., mandrel shape, filament design, insertion tools, and methods) can be tailored to suit each application. For example, some filaments can be preferentially wound in certain locations such that certain aspects of the tamp/implant have different diameters or geometries. For example, additional filament can be wound onto one location on the mandrel in order to create a greater thickness at that location or a greater concentration of certain mechanical or chemical or drug features at that location.

The use of the assembly device may be as a temporary instrument (as a tamp or distractor) or as a permanent implant. In instrument embodiments, in situ spool creation provides a bone tamp having a highly controllable geometry that displaces a volume of tissue and possibly creates height restoration within the vertebral body. Upon removal of the tamp, the resulting void may then be filled with bone cement. In implant embodiments, the spool may be left behind as an implant. Cement may then be injected into or around the spool to further stabilize the implant.

### DESCRIPTION OF THE FIGURES

FIGS. 1a-b disclose side and top views of a mandrel and guide and filament of the present invention.

FIGS. 1c-e disclose the sequential winding of a filament upon a mandrel to create a spool.

FIGS. 2a-b disclose side and top views of a spool of the present invention.

FIG. 3 discloses a cross-section of a spool displacing cancellous bone within a vertebral body.

FIGS. 4a-b disclose respective side views of a mandrel and spool of the present invention within a vertebral body.

FIGS. 5a-c disclose various embodiments of the present invention comprising flags.

FIGS. 6a-c disclose various embodiments of the present invention comprising stars.

FIG. 7 discloses a spool wherein the filament has a plurality of cross-sections.

FIG. 8 discloses a spool wherein the filament has a “+” cross-section.

FIG. 9a discloses a cross section of a spool wherein the mandrel has ribs.

FIG. 9b discloses a spool in which the filament diameter is at least 50% of the height of the ribs.

FIG. 9c discloses a spool wherein the mandrel has 5 ribs.

FIG. 10 discloses a bulging spool.

FIG. 11a discloses a side view of a lordotic spool.

FIG. 11b discloses a side view of a concave spool.

FIGS. 12a-d disclose various views of a guide of the present invention.

FIGS. 13a-f disclose various views of a spool of the present invention having deployable wings.

FIGS. 14a-b disclose respective side views of a mandrel and spool of the present invention between adjacent spinous processes.

FIG. 15a discloses a side view of a spool within a vertebral body.

FIG. 15b discloses a front view of a pair of spools within a vertebral body used for scoliotic correction.

FIG. 16 is a side view of a frustoconical spool of the present invention.

FIGS. 17a and b disclose a deflected spool of the present invention.

FIG. 18 discloses a top view of a device of the present invention having two spools.

FIG. 19 discloses a top view of a device of the present invention having two filament guides.

FIG. 20 discloses a device of the present invention wherein the filament guide has two filament openings.

FIGS. 21 a and b disclose a first filament having a discontinuity.

FIG. 22a discloses a second filament having a long discontinuity.

FIG. 22b discloses a cross-section of a spool having closely spaced discontinuities.

FIGS. 23a and b disclose chain link filaments of the present invention.

FIGS. 24a and b disclose embodiments of an offset device of the present invention.

FIG. 24c discloses a cross-section of FIG. 24b.

FIGS. 25 a and b disclose cross-section and side views of a first device of the present invention.

FIG. 26 discloses a cross-section view of a second device of the present invention.

FIG. 27 discloses pedicular cross-section of a device of the present invention.

FIGS. 28a and b disclose bottom and side views of a distal end of devices of the present invention

FIG. 29 discloses a deflected cannula of the present invention.

FIG. 30 discloses a cross-section of a spool of the present invention wherein the cannula acts as a spring.

FIG. 31 shows a device of the present invention implanted through an extrapedicular apoproach.

### DETAILED DESCRIPTION OF THE INVENTION

For the purposes of the present invention, a “mandrel” is defined as an elongated mechanical device around which a material is wound or wrapped. Similarly, a “filament” is defined as a substantially linear, flexible, mechanical device that can be wound or wrapped around the mandrel. The “spool” is the assembly produced when the filament is wrapped around the mandrel. A “turn” is the portion of the filament that circumscribes the mandrel once. A “winding” is the wound filament.

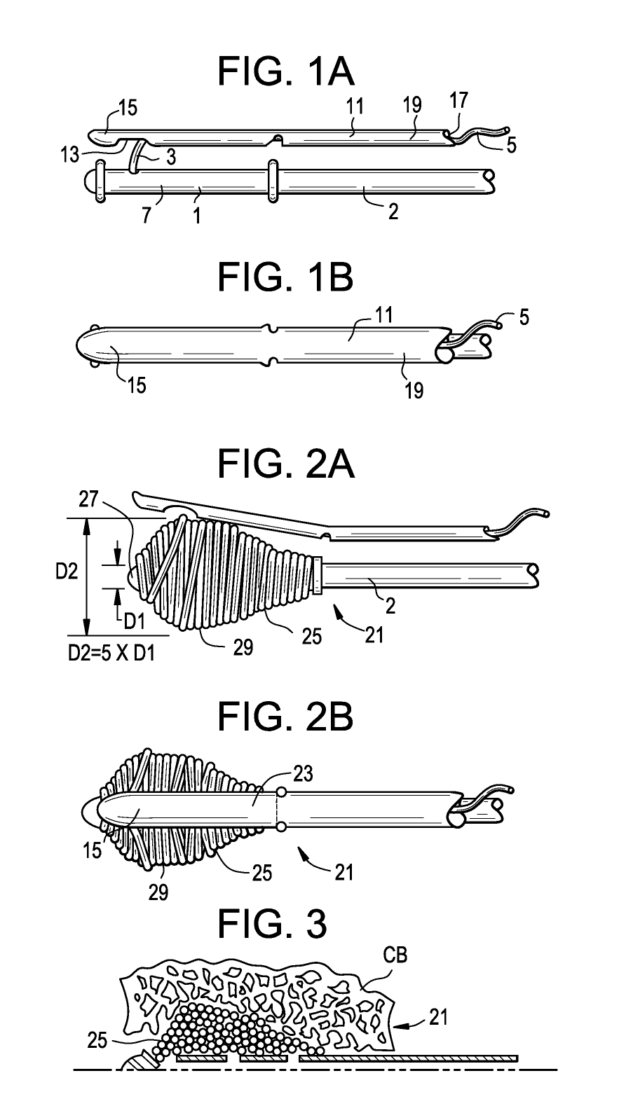

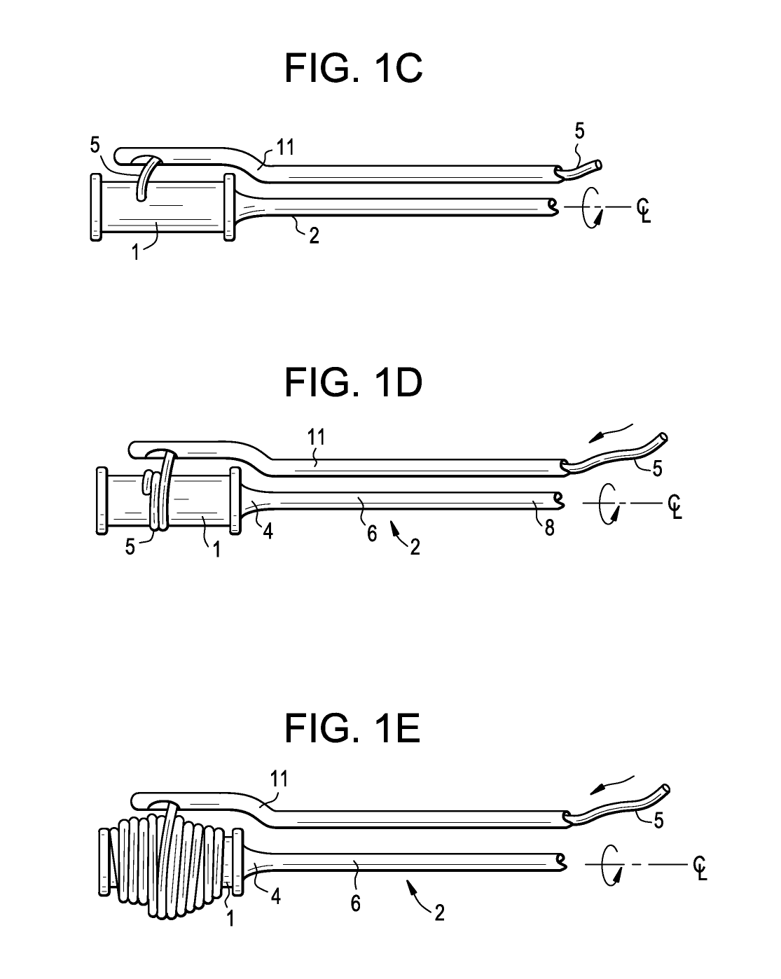

In general, to create a spool, the clinician winds a filament around a mandrel. In some embodiments, the mandrel is first inserted into the tissue space using a longitudinal insertion tool. As shown in FIGS. 1a-e, the present invention may be carried out with an insertion tool comprising a longitudinal rod 2 comprising i) a distal end portion 4 comprising a mandrel 1, ii) an intermediate shaft 6, and iii) a proximal end portion 8. The filament and mandrel may be joined prior to mandrel insertion, assembled in situ, or created in situ. Next, rotation of the insertion tool about its longitudinal axis rotates the mandrel core to wind the attached filament around the mandrel. In another embodiment, the filament is wound around a stationary mandrel. As filament material is added around the mandrel, the geometry of the assembly may change in a predetermined manner due to an orderly accumulation of filament material on the mandrel. However, in other embodiments, the geometry may change in a random or non-predetermined manner whereby filling occurs first in the cancellous portions having the weakest resistance to being filled.

Now referring to FIGS. 1a-1b, there is provided a mandrel 1 having a first end 3 of a filament 5 attached thereto. In this case, the filament is attached to a distal portion 7 of the mandrel. The filament also is passed through tubular filament guide 11 having a distal opening 13 formed in its distal end portion 15 and a proximal opening 17 formed in the proximal end portion 19. Preferably, the mandrel, filament and guide are inserted into the vertebral body at the same time. As the mandrel begins to turn and wind filament thereupon, the location of the distal opening guide is manipulated so that the filament is preferentially deposited upon the mandrel at a desired location. It may be possible to deposit multiple filaments on the mandrel from one guide or multiple guides.

FIGS. 1c-1e disclose the gradual winding of the filament 5 upon the mandrel 1. FIG. 1c discloses the initial locations of the mandrel and filament prior to mandrel rotation. FIG. 1d discloses the beginnings of the spool creation, with about three turns of the filament 5 being wound upon the mandrel 1. FIG. 1e discloses a finished spool 21.

Now referring to FIGS. 2a and 2b, there is provided a spool 21 comprising a mandrel 23 and a wound filament 25. The wound filament is located essentially exclusively upon the distal end portion 27 of the mandrel. The winding bulges in its center region 29 due to extra filament being deposited in that region. This bulging region provides restoration of vertebral body height.

Now referring to FIG. 3, there is provided a cross section of a spool 21 displacing cancellous bone CB within the vertebral body. This displacement, caused by the gradual building of the wound filament 25, should result in height restoration within a fracture vertebral body.

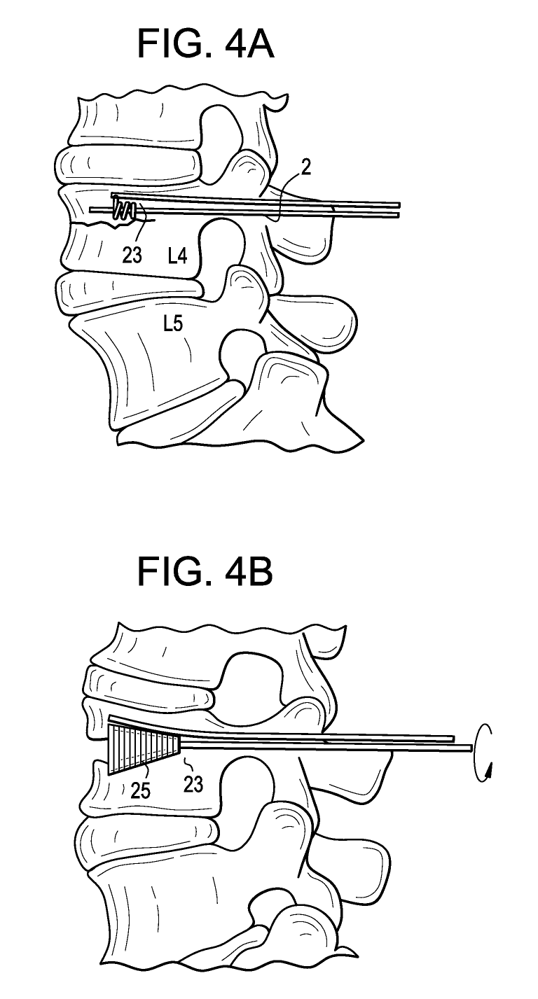

Now referring to FIG. 4a, there is provided a cross-section of a longitudinal rod 2 comprising a mandrel 23 of the present invention inserted into a fractured vertebral body. Now referring to FIG. 4b, there is provided a cross-section of a spool 21 of the present invention with a full winding 25 disposed within the fractured vertebral body to restore height.

The filament could be a thin string, or it could be a material with volume or three-dimensional geometric characteristics (such as a chain).

Wrapping a cable, cord, chain, tether, braid, linear assembly of parts or other types of filament around the perimeter of a mandrel is the preferred means of increasing the effective thickness of the mandrel. The filament is preferably a chain, belt, fiber, or a tether. The geometric cross-section of the filament imparts certain characteristics to the implant, tamp, or distractor. A small-diameter filament creates spool with smaller pores whereas large-diameter or complex cross-section filament creates a spool with larger pores. When pores become very small, the material may approximate a solid. The clinical benefit of having pores may be found in tissue incorporation (osseointegration), fluid wicking, or cement interdigitation. The degree to which fluids will permeate the spool depends on spool geometry, porosity, fluid viscosity, surface chemistry/energy, capillary pressure, and surface tension. Viscous fluids might not penetrate materials with small pores unless there is preferential chemical attraction between the material and filament.

The choice of filament material may also impart certain desirable characteristics in the device. The material could have a low coefficient of friction or be lubricious to improve the sliding action of the spool against the native tissues. The filament material could be abrasive to act as a rasp or to prevent unwinding of the spool assembly. The filament material could be adhesive to prevent unwinding of the spool assembly. The filament material could have chemical, biological, or geometric features therein to affect local biological changes. For example, the filament may have a geometry such that winding of the filament could create a certain controlled porosity suitable for osteoinductive bone ingrowth, generally in the range of about 10-500 micron pore size. The filament material could be made to be electrically, thermally, or optically conductive for energy transfer. For example, metallic particle additives could make the filament electrically conductive and/or thermally conductive. The filament material could be chemically reactive with subsequently-added materials (like curing agents or adhesives) that are injected into or around the spool. The filament material can contain radiopaque agents that allow the clinician to radiographically monitor the placement of the device.

In some embodiments, the filament material can contain discontinuities (such as continuous or discrete porosity), discreet accumulations of the base filament material (such as barbs), or material additives (such as flags, stars, or radio-opaque markers).

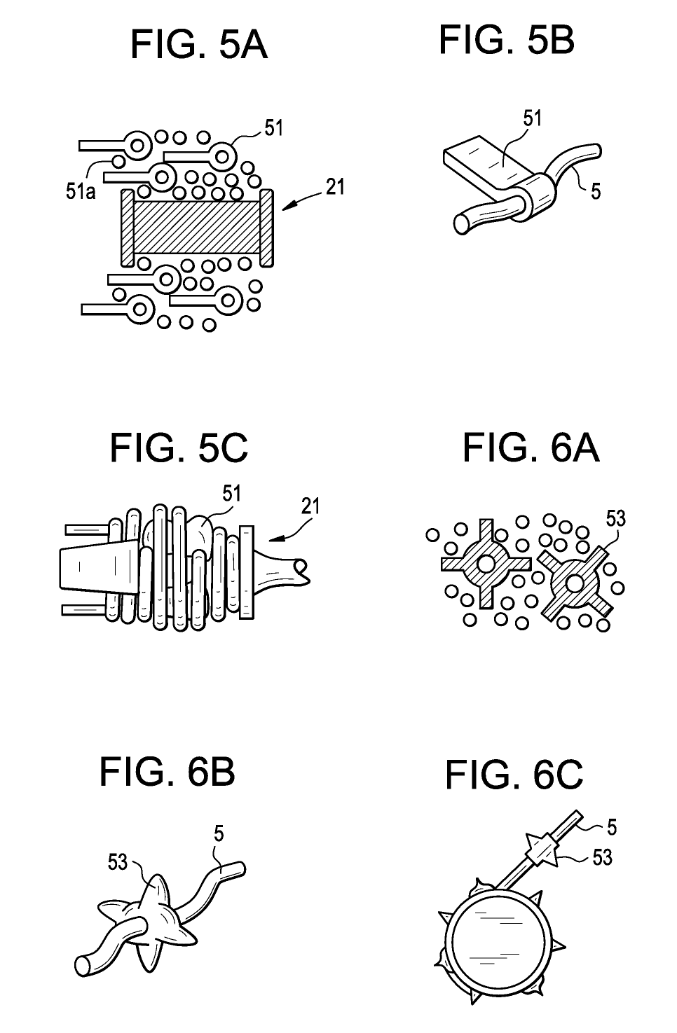

These discontinuities, accumulations and additives can be used to stabilize the spool as it winds. For example, flags of material are shown in FIG. 5a. Now referring to FIG. 5a, there is provided a cross section of a spool 21 whose winding comprises flags 51. FIG. 5b discloses a filament 5 having a flag 51 attached thereto. Now referring to FIG. 5c, there is provided a side view of a spool 21 whose winding comprises flags 51. Incorporation of such flags onto the filament could enable the spool to form beyond the end of the mandrel. For example, FIG. 5a shows windings 51a extending beyond the mandrel end. The flag materials extending from the end of the mandrel within the spool could cut tissue deep to the spool. In other embodiments, the flags could contain catalysts (such as enzymes) and thereby enable catalytic reactions to take place. The flags could contain bone growth promoting agents, and thereby act as osseointegrating- or biologically active-surfaces. The winding geometry within the spool can be modified by using the discontinuities to generate pores of specific size and shapes. The outside spool windings can be constructed from filaments with specific textures, surface chemistry, porosity, surface energy, etc.

Star-like inclusions on the filament could act as rasps as the material is drawn onto the spool and rotated as the spool winds. Now referring to FIG. 6a, there is provided a cross section of a spool whose winding comprises stars 53. FIG. 6b discloses a filament 5 having a star 53 attached thereto. Now referring to FIG. 6c, there is provided an axial view of a spool 21 whose winding comprises stars 53. The embodiment of FIG. 6c has an advantage in that the stars 53 may act as rasps that cut the bone tissue as the mandrel rotates, thereby helping bone displacement.

The discontinuities in the filament could enable the user to mark the amount of filament material deposited on the spool.

One filament characteristic is its cross-sectional geometry. Although a circular filament cross-section is contemplated as within the scope of the present invention, it may possess a number of shortcomings. For example, the successive layering of circular turns of the filament produces an inefficient packing density and creates small voids. In addition, adjacent circular turns of the filament can slip one over the other within the spool in response to stress, thereby creating instability. It is believed that selecting the cross-section of the filament to be a rectangular or flat ribbon shape will create a much more efficient packing density, eliminate voids, and prevent individual turns from slipping one over the other (if packed properly). Similarly, the cross-section could be substantially hexagonal to theoretically create an ideal packing efficiency, help in winding efficiency, eliminate voids, and resist sliding of adjacent turns one over the other. Alternatively, the cross-section could be designed to avoid perfect packing, or intentionally create voids, gaps, or enables or disables adjacent turns from slipping one over the other. Such a cross-section could preferably be that of a chain-link structure, or a star-shaped section, or a geometry that is randomized in cross-section along the length of the filament. Such structures create a porosity when packed which may enhance tissue ingrowth or lower stiffness. The chain link structure also allows relatively brittle materials such as acrylics (such as PMMA) to be used as the filament. Finally, the filament could comprise an assembly of geometries in linked particles (like a chain) or weaves, yarns, multi-component assemblies, knotted groups, or combinations thereof. In general, mechanical properties of materials tend to optimize with smaller geometries. For example, actual material stiffness approaches theoretical material stiffness (determined by atomic bonding, weak forces, polymer-chain alignments, or crystalline structures). Thus, assemblies of small geometry links or filaments can be used to tailor the ultimate spool mechanical properties.

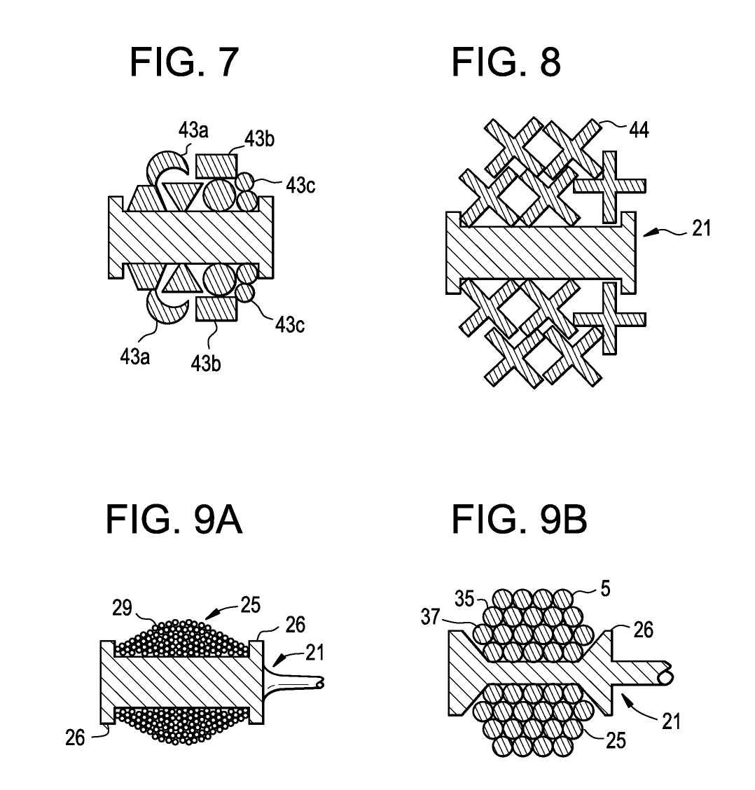

Now referring to FIG. 7, there is provided a spool 21 whose filament has a plurality of cross sectional shapes 43a, 43b and 43c. The advantage of providing a cross-section with sequentially different shapes is that the resulting winding will have a significant porosity. Also, pore sizes can be varied and/or diversified within the spool by changing filament cross-section.

Now referring to FIG. 8, there is provided a spool 21 whose filament has a “+” cross sectional shape 44. The advantage of this “+” shape is that the winding so produced has a substantial porosity, thereby enabling cement to be easily flowed therethrough. A spool constructed of such a filament can pack efficiently (resulting in a low porosity) or threading fibers one over the other can enhance resistance to fiber slippage within the spool because of higher normal contact forces between windings attributable to point contacts between filaments.

Therefore, preferably the filament has a substantially polygonal cross-section. In some embodiments, the polygonal cross-section is substantially a hexagon (so as to approach perfect packing) In some embodiments, the polygonal cross-section is substantially rectangular, and more preferably, its the width is at least two times its height (so that it begins to take on a ribbon appearance). In some embodiments, the wound filament defines a porosity of no more than 40 vol %, preferably no more than 20 vol % in the wound region, most preferably no more than 10 vol %.

Alternatively, the filament cross-section can be designed to prevent its efficient packing in the spool, or to enable packing at one location and changed at another location. Such a configuration could be similar to a conventional chain. When circular chain links wrap around the spool, they create a porous structure with high resistance to slipping between the individual windings. Such a porous structure is beneficial for initial in situ slip resistance, tissue ingrowth, and/or biodegradation. In addition, the porous structure of the chain link may have the effect of decreasing the overall stiffness of the device, thereby reducing the likelihood of adjacent vertebral body fracture.

In some embodiments, the filament is a chain link made of an acrylic material (such as PMMA). The chain link design allows a bendable filament to be made from a relatively brittle (acrylic) material while avoiding concerns of bending-induced breakage. The chain link design also provides predetermined contact points amenable to sintering.

In some embodiments involving a flat ribbon filament, voids may be incorporated into the filament. Such a ribbon filament with voids would not only provide improved filament packing and reduce slippage between turns, but also provide structural voids for tissue ingrowth, imbibing of fluids, biodegradation, and/or drug release. The ribbon void fraction could then be used to contain another material such as a drug, biomaterial, nutrient, ingrowth surface, ceramic, radiographic marker, etc. Such a ribbon material could contain large or small voids that provide space for a resin or binder to be injected or imbibed into the structure. Such a resin or binder infused device would form a composite structure with improved geometric features, local stability, bone fragment capture, osseointegration, or mechanical properties. The resin or binder could be a component of the filament (such as a coating), it could be injected into the tissue space prior to device insertion, or it could be injected into the device before/during/after deployment of the mandrel and creation of the spool. For example, in some embodiments, there is provided a flat ribbon-like chain with a void for containing ceramic bone void filler. In other embodiments, the voids of the ribbon could contain a reactant that reacts with PMMA.

A filament with changing cross-sectional properties could also possess highly modified chain links, whereby the link is geometrically designed to possess a particular net cross-sectional area, voids, chain-linking features, drug release features, and/or mechanical features (such as tynes).

Other filament modifications may include snap-on features to attach to other chain links. Still others may include tynes extending from one or more surfaces to pierce chain links in subsequent windings, wherein the tynes either provide additional mechanical stability or pierce drug or chemical release reservoirs upon winding the ribbon chain.

In some embodiments, there can be two or more filament feeds or sources. In some embodiments, the filament could be inserted in a random fashion into the tissue space prior to insertion of the mandrel, and this randomly deposited filament could then be organized in situ by a rotating tyned mandrel (similar to how noodles are organized by twisting a fork).

Typically, the mandrel has a cylindrical shape so that it is easily rotated about the longitudinal axis of the longitudinal rod of the insertion tool.

In some embodiments, the mandrel comprises a substantially cylindrical outer surface and at least one rib extending from the outer surface. Preferably, the rib extends completely around the circumference of the mandrel. Preferably, the mandrel comprises at least two ribs extending from the outer surface, again completely around the circumference of the mandrel. More preferably, the mandrel comprises a distal end portion and a proximal end portion, wherein a first rib extends circumferentially from the distal end of the mandrel and a second rib extends circumferentially from the proximal end portion of the mandrel. In this condition, the ribs act to retain the filament upon the mandrel (i.e., prevent the filament from sliding off the ends of the mandrel) and help build thickness in the spool. In some preferred embodiments, the filament has a thickness and the rib has a height, and the thickness of the filament is less than the height of the rib (so that the rib is tall enough to retain the filament). More preferably, the thickness of the filament is more than 50% of the height of the rib (so that the overall height of the mandrel is small). In some embodiments, the rib is a static element. In others, it is deployable. The rib could be deployed from the end of the mandrel in situ (like a balloon, compressed elastic material, deformed tines, filament splay, etc.). Alternatively, a length of the mandrel could reduce in diameter in situ. Deformation by rotation, elastic extension, and application of a vacuum to a deformable surface are mechanisms to reduce the mandrel diameter thereby creating “ribs” by subtraction rather than addition.

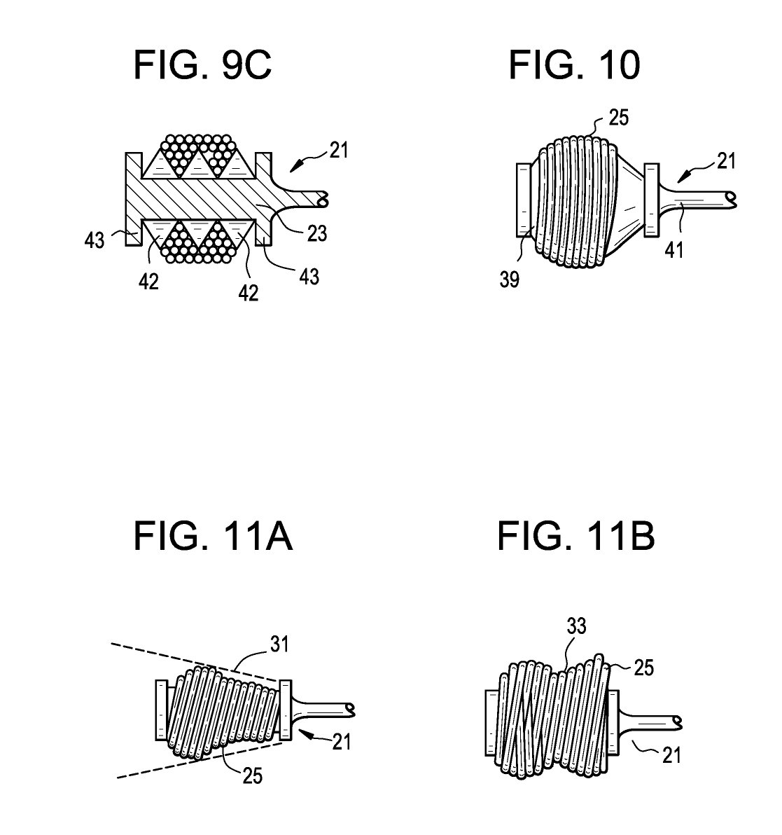

Now referring to FIG. 9a, there is provided a cross section of a spool 21 in which the wound filament 25 is located between mandrel ribs 26 and bulges in its central region 29.

Now referring to FIG. 9b, there is provided a spool 21 in which the diameter of the filament 5 in the winding 25 is more than 50% of the height of the mandrel ribs 26. The filament at a higher level 35 of the winding deposits between the filament of the immediately lower 37 level. This produces a stable construct.

Now referring to FIG. 9c, there is provided a spool 21 whose mandrel 23 has a series of ribs 43, five in this case. The advantage of the series of ribs is predetermined filament spacing, thereby providing construct stability.

In some embodiments of the present invention, operation of the device may desirably fracture or re-fracture bone as may be required in the treatment of compression fractures. The spool's creation of a bone cavity and displacement of bone material should enable the re-fracture of a collapsed vertebral body, thus restoring the vertebral body to its previously intact height. The device of the present invention can include features specifically incorporated to enable vertebral body refracture. One such feature is a cannulation of the mandrel. This cannulation enables the deployment therethrough of an anterior release blade or vibratome (such as an ultrasonic cutter) to sweep the tissue anterior to the end of the implant. Like a fan, this anterior release vibratome can create a new fracture plane as the mandrel is inserted into the vertebral body. If this is done bilaterally (through both pedicles), the two independent release planes will define the fracture surface once the mandrel accumulates filament and expands the space. In some embodiments, the vibratome reaches to the anterior cortical shell and fractures the cortical shell of the target vertebral body. The vibratome could be a simple vibrating flail (wherein the clinician can shake the base of an unsupported beam, resulting in a vibratome flail capable of pulverizing a volume of tissue surrounding the excited beam/flail).

In some embodiments, the mandrel has a longitudinal bore therein that opens either onto a side of the mandrel or onto the distal end face of the mandrel. In some embodiments, the mandrel is a hollow tube with at least one side opening. This mandrel can be attached to a lavage/suction instrument so as to act as a lavage/suction tube to facilitate tissue removal. Suction irrigation is a means to prevent profuse bleeding, to stimulate a healing response, to prevent fat embolism, to deliver local bone aiding agents, to remove clots, and to prepare the tissue for distraction and healing (such as an epinephrine and lidocaine lavage to prevent bleeding and pain). Likewise, the irrigation fluid could enable deployment of the wound filament by lubricating the local tissues. In preferred embodiments, the lavage fluid is saline. However, alternatively, the lavage could also be a cement or sealant (such as PMMA, cyanoacrylate, hyaluronic acid, calcium sulfate, fibrin glue/sealant, bis-TEG/GMA, etc.).

In some embodiments, the lavage fluid is set at a temperature above the glass transition temperature (Tg) of the acrylic filament. In some embodiments, the lavage fluid is set at a temperature 5° C. above the Tg of the acrylic filament. In some embodiments, the lavage fluid is set at a temperature 10° C. above the Tg of the acrylic filament. This heated lavage enables sintering of the in situ deposited acrylic filament material.

The cannulation can be open to the environment in order to achieve fluid communication with surrounding tissues, or it can be closed and thereby form a self-contained circulatory system within the mandrel. Openings along the length of the mandrel can enable controlled leakage into specific regions of tissue. The openings can be shaped to enable pulse lavage and/or water-jet lavage of local tissues. A forward opening of the mandrel's cannulation may be used to hydrodissect the fracture plane during insertion and deployment.

In some embodiments, the mandrel may be removed from the final assembly so that only the wound filament remains as the implant. The mandrel could be biodegradable. The mandrel could be relocated to a new tissue location for continued filament winding. The mandrel could be radio-opaque or contain ultrasonic signal generators.

Now referring to FIG. 10, there is provided a spool 21 in which the distal end portion 39 of the mandrel has a greater diameter than that of the proximal end portion 41 of the mandrel. The advantage of this design is that a uniform winding creates a larger copy of the original mandrel. That is, a spool that starts out with a 6 mm distal diameter and a 3 mm proximal diameter can produce a finished spool with 11 mm distal and 8 mm proximal diameters with the addition of 2.5 mm of winding material.

In some embodiments, a large diameter mandrel can be collapsed after creating a spool, thereby creating a large void within the spool. The benefits of such a void enable easy insertion of subsequent materials (PMMA cement, calcium bone substitutes, bone graft, large volume drug depot). Also, the large void can be left unfilled in such a way as to make the spool assembly more mechanically compliant. Increased implant compliance may retard vertebral end-plate fracture or adjacent level vertebral body collapse due to an overly-stiff vertebroplasty implant. Alternatively, the large diameter mandrel could be used to deliver a significant thermal mass for sintering or other therapeutic benefit. Finally, creating a large diameter mandrel enables necking of such a mandrel to the thin proximal shaft, which allows for torsional shaft fracture for shaft removal from the ultimate spool assembly.

In some embodiments, the mandrel is relatively flexible and the guide is relatively stiff. In these embodiments, the spout of the guide is located immediately lateral to the flexible mandrel. As deposition of the filament upon the mandrel continues, the winding grows. However, because the guide is stiff and the mandrel is flexible, the side feeding location of the guide pushes the growing mandrel medially.

Without wishing to be tied to a theory, it is believed that the flexible mandrel/stiff guide will possess at least two extra advantages. First, when the guide is placed lateral to the mandrel, the displacement of the mandrel will be medially away from the cortical wall of the vertebral body. This medial displacement reduces the chances that growth of the spool will cause vertebral body wall blowout. Second, the medial displacement of the spool may cause the spool to be located sufficiently central so that only a single spool would be required in a vertebral body. This unilateral capability would amount to a significant cost savings for the hospital.

In preferred flexible mandrel embodiments, the stiff guide has a spout that is located proximal to the distal end of the guide. Locating the spout in such a proximal location allows the guide to continually contact the entire axial length of the winding during spool creation.

Mechanical components that can create a flexible but controllable mandrel are known in the art. One such mechanism is a series of “universal joints” that enable rigid rotation of a shaft with concomitant deviation in shaft segment longitudinal axes. Another mechanism is a machined spring concept that enables the mandrel to exhibit high torsional stiffness with high cantilever beam compliance. Finally, a wound filament spring mandrel (similar to those used in flexible drive shafts) would enable mandrel rotation about a nonlinear axis.

The spool shape can be designed based on mandrel and filament requirements. The spool can be formed within a bag or enclosure (such as the Perimeter™ vertebroplasty device available from DePuy International, Leeds, UK).

A spool grows in effective diameter as a filament is repeatedly wound around the mandrel. If the mandrel turns on its longitudinal axis, it will wind the filament around its core. As the mandrel is rotated, turns of filament wrap around the core adding mass to the spool and changing the spool's effective geometry. With each turn of filament wound around the mandrel, the spool grows in effective diameter.

In some embodiments, the diameter of the resulting spool is at least twice the diameter of its mandrel component (excluding ribs). Preferably, the diameter of the resulting spool is at least three times the diameter of its mandrel component, more preferably at least four times, most preferably at least five times.

In some embodiments, the filament can be preferentially applied to the mandrel upon the distal portion of the mandrel, thereby creating an implant whose winding thickness is greater distally. The resulting implant preferentially restores vertebral body height in the anterior portion of the vertebral body, thereby providing desired lordosis to the patient.

Now referring to FIG. 11a, there is provided a spool 21 in which the wound filament 25 is preferentially deposited so as to form lordotically angled surface 31. This lordosis is desirable for restoring the physiologic shape of the vertebral body within the cervical or lumbar spine.

Now referring to FIG. 11b, there is provided a spool 21 in which the wound filament 25 is preferentially deposited so as to form a concavely angled surface 33.

In some embodiments, the filament is applied to the mandrel using a feeding mechanism. In some embodiments, the feeding mechanism is a separate device (like a cannula), while in others it is integrated into the mandrel-turning tool. In still others, it is a permanent feature of the resulting implant. The feeding mechanism may be used to selectively place filament on the mandrel, thereby creating novel or customized implant shapes in situ.

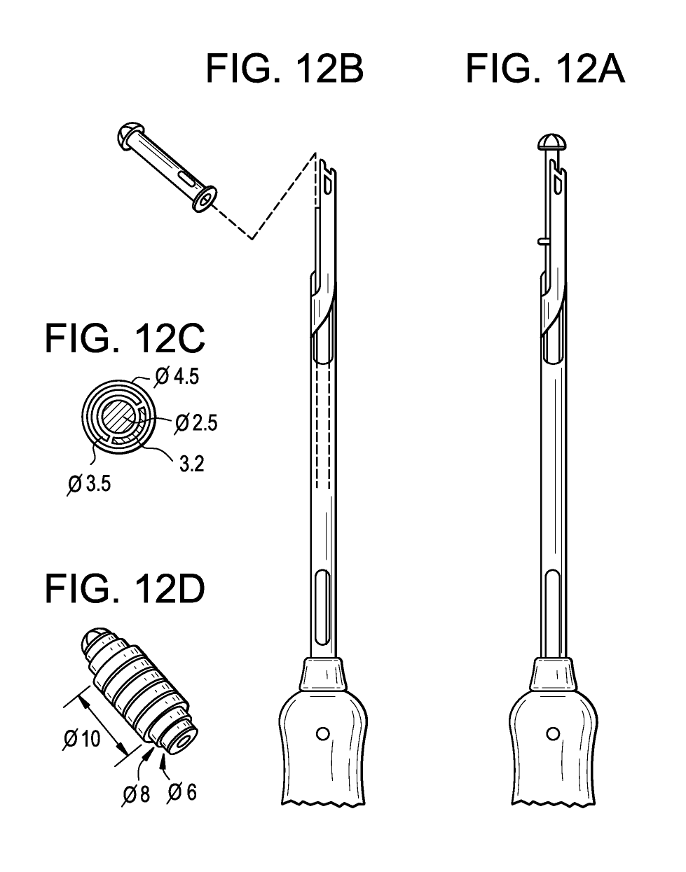

Now referring to FIGS. 12a-d, there is provided a guide instrument for depositing the filament upon the mandrel.

The insertion tool 201 accepts a mandrel 203 upon its distal end portion 205 via a splined connection 207. Mandrels can be provided with various lengths, widths or cross sections, as determined by surgical application. The filament 209 is fed through the tool including the eye of the feeder arm and then connected to the mandrel. The filament connection to the mandrel can be an eye or throughhole, a clamp, or a wedge. Alternatively, it can simply held in place while the user pre-winds a first layer. The insertion tool, with loaded mandrel and connected filament, is now inserted into the surgical site.

The handle of the tool and a proximal portion of the shaft remain exposed proximal to the surgical entry point. A main positioning handle is manipulated to locate the mandrel in-situ. A secondary knob, lever or squeeze trigger is used to rotate the inner cylindrical shaft, which rotates the mandrel, causing the filament to wind thereupon. The filament may originate from either a spool within the secondary knob, from a cartridge loaded within the tool, or from an external spool/card not connected to the tool.

The feeder arm is the distal portion of an inner tube of the insertion tool. The cylindrical axis can turn freely within the inner tube. As the mandrel is rotated, the proximal portion of the inner tube is directed to advance along the mandrel in predetermined or surgically-selected patterns. For example, the feeding arm can be advanced and retracted to produce the spool of FIG. 12d. The proximal end directing portion is preferably a roller-cam mechanism seen commonly in the textile industry, but could be any form of mechanical linkage, exchangeable profile/card, or programmable actuator.

When the appropriate amount of filament has been applied, the surgeon can cut the filament and slide out the inner tube, followed by the cylindrical splined axis. Alternatively, the filament can be marked with indicia of spool diameters. When the marking for the desired size appears in the proximal window, in plain view, the surgeon can cut the filament proximal to the surgical entry. A few more turns of the secondary knob advances the remaining portion of the filament off the feeder arm, thereby freeing up the tool for extraction.

In some embodiments, the mandrel is surrounded by a delivery cannula having distal pivoting “wings”. A winged mechanism at the end of the cannula can be used to prevent filament from coiling beyond the end of the mandrel, thereby frustrating the spool creation. In use, the spool is generated within the cannula. As the spool grows in diameter, the growth of the filament winding gradually deploys the cannula wings. After adequate distraction has been created, the mandrel can be removed or left behind, cement can be injected, and/or the cannula can be detached.

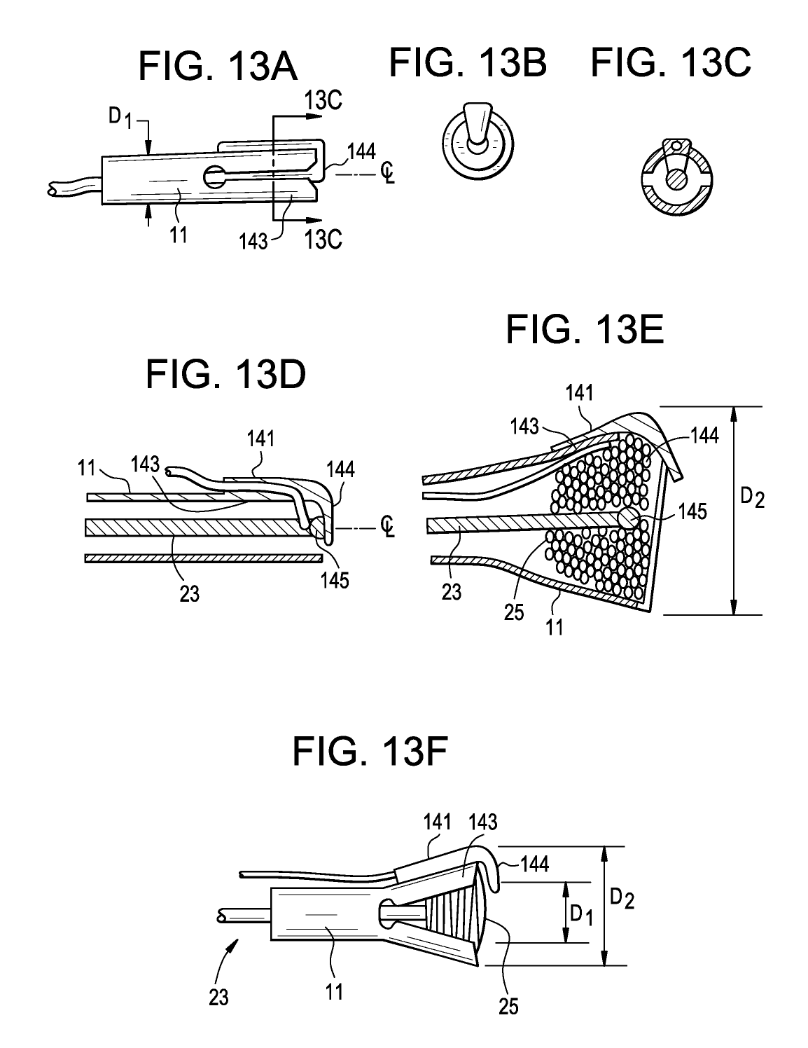

Now referring to FIG. 13a-f, there is provided a device of the present invention comprising a mandrel 23 surrounded by a delivery guide 11, wherein the guide comprises deployable wings 141 extending from the distal end portion 143 of the guide. A lip 144 is formed at the distal end of the deployable wing and extends radially inward towards the distal end 145 of the mandrel. FIGS. 13e and 13f show that as the winding 25 grows, the lips 144 of the deployable wings 141 help retain the winding upon the mandrel 23.

Another embodiment of the device winds filament onto a mandrel contained within an envelope. The envelope can be used as a bearing surface between the filament and the bone, thereby protecting the winding upon the mandrel. The protective envelope enables an idealized winding environment for the spool creation by preventing trabecular bone spurs and struts from imparting uncontrollable and unpredictable forces upon the windings. Alternatively, the envelope may be a chemical/hydrostatic barrier that prevents the filament from contacting tissue while winding upon the mandrel. If the envelope is left in situ, it becomes part of the implant. In such situations, it may be made of a biodegradable material. Removal of the envelope may be desirable to enable the filaments to interface with surrounding tissues, which is especially important if the filaments provoke chemical or biological responses with surrounding tissues.

In some envelope embodiments, the spool is formed within a mesh. In some embodiments, the mesh is substantially similar to that used in the DePuy International Perimeter™ product for use with Confidence™ PMMA cement systems (available from DePuy International, Leeds, UK). Current challenges of traditional vertebral augmentation include an inability to achieve height restoration and an imperfect control of ultimate PMMA location. Forming the spool within the mesh facilitates height restoration, modulates friction during spool creation, contains the cement after injection, and controls the location of the cement after spooling.

Preferably, the insertion tool of the present invention holds and rotates the mandrel about the mandrel's cylndrical axis. The tool holds, positions, and applies filament. The tool enables an in situ winding process. The tool enables insertion/removal of the assembly. The tool enables placement of the envelope. The tool enables orientation of the device. The tool can contain one or more lengths and/or sources and/or types of filament. The tool enables the winding and unwinding of the filament from the mandrel. In some embodiments, the tool resembles a fishing reel. In this embodiment, an external spooled filament feeds onto the mandrel with an attached drive mechanism. The drive mechanism can be disconnected from the mandrel and attached to the external spool to enable “unspooling” of the mandrel. In this way, a power-driver can be used to spool and/or unspool the assembly.

The filament guide directs the application of the filament upon the mandrel. In some embodiments, the filament guide comprises a cannula through which the filament is fed onto the mandrel. In use, the distal end of the cannula tool (or “spout”) is placed against a side of the mandrel, and the surgeon changes the longitudinal location of that spout in order to change the location of the mandrel onto which the filament is fed. In some embodiments, the surgeon can produce tension in the filament so that it is applied tautly to the spool. The filament can be fed from within the spool or from outside the spool. An internal filament guide can be inserted through a cannulation in the mandrel. The internal filament guide would then place filament at a distance to the mandrel surface thereby enabling spool creation.

The device of the present invention may be used in other spinal applications beyond the vertebral body augmentation application.

In some embodiments, the device of the present invention may be used as an interspinous process spacer. In such embodiments, the mandrel is inserted between the two spinous processes of the target functional spinal unit. One or more saddles that bear upon the interspinous processes can be inserted before, during, or after the insertion of the mandrel. The saddles bear upon the upper and lower surfaces of the spool and distribute the load and material along the edge(s) of the spinous processes. The filament feeding device can be inserted before, during, or after the mandrel and/or saddles. Upon rotation of the mandrel, filament will accumulate between the spinous processes and/or saddles, thus creating a well-controlled distraction force. Eventually, an appropriate distraction distance between the spinous processes will be achieved. In some embodiments, the mandrel can then be removed. Cement can be used to further stabilize the spool and/or spool-saddle implant. In some embodiments, no saddles are used so that the upper and lower portions of the spool bear upon the adjacent spinous processes.

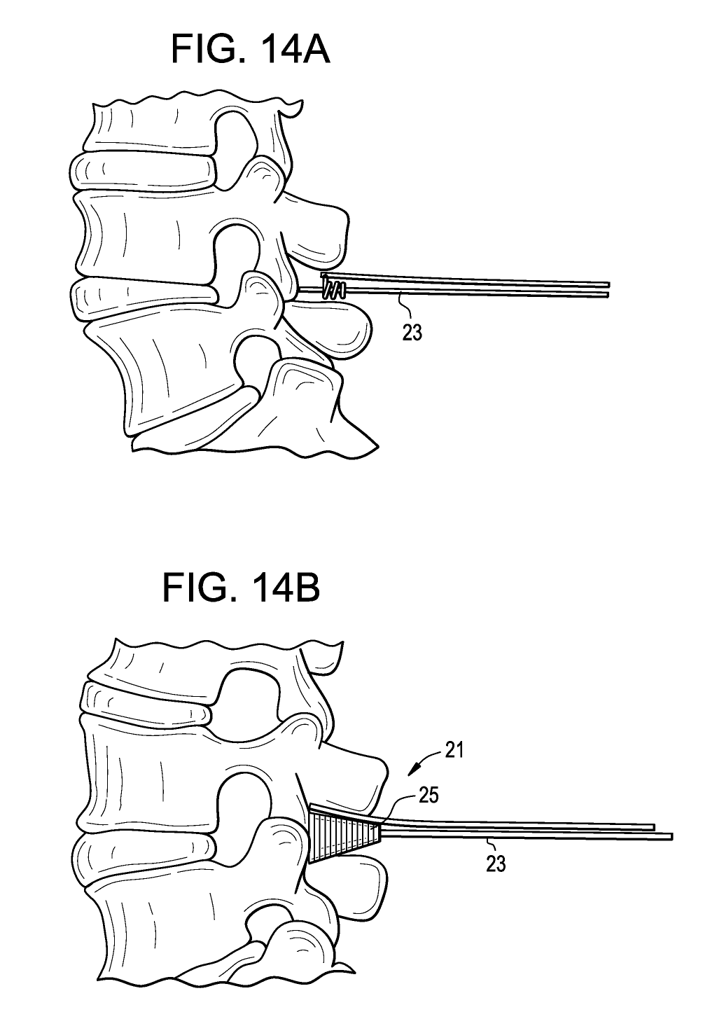

Now referring to FIG. 14a, there is provided a cross-section of a mandrel 23 of the present invention inserted between adjacent spinous processes. Now referring to FIG. 14b, there is provided a cross-section of a spool 21 of the present invention with a full winding 25 disposed between adjacent spinous processes.

In some embodiments, the device of the present invention may be used as an interbody device within the intervertebral disc space. In such embodiments, a mandrel is inserted into the interbody/intersomatic space before or after discectomy, and the mandrel is spun so that filament material is wound onto the mandrel. In some embodiments, the action of winding the mandrel with filament can effectively rasp or remove the local discal/nucleus pulposus material (and thereby act as a discectomy device). If left in situ, the assembly can act as a nucleus pulposus replacement device. If unwound and removed, the assembly will leave a tamped and/or excised interbody space. If the assembly is allowed to wind substantial amounts of filament, the diameter of the assembly could grow larger than the existing distance between vertebral endplates. In this application, the assembly acts as a distractor. If this distracting body is left in situ, the assembly acts as an interbody device. In some embodiments, the windings could comprise “distractor” filaments in some locations and “implant” filaments in others (MF: please explain). For example, certain spool assemblies in a tissue space could be made of high strength steel filament with very low frictional resistance to deployment (distractor embodiment) while other spool assemblies in the tissue space can be made of PEEK filament (implant embodiment) that might have non-ideal distraction characteristics but highly desirable mechanical and biocompatibility characteristics. Assemblies of distractor and implant spools can be used in one procedure in one tissue space. In other embodiments, assemblies of filaments could be employed (e.g., drug-eluting filament windings on some mandrels and load-bearing implant windings on other mandrels, or on the same mandrel). Another embodiment of the device winds filament onto a mandrel contained within an envelope. The envelope can be used as a bearing surface between the filament and vertebral endplates, thereby enabling winding of the mandrel. Alternatively, the envelope could be a chemical/hydrostatic barrier that prevents the filaments from bodily contact while winding the mandrel. Alternatively, the envelope can act as a mechanical cradle while winding the filament onto the mandrel. If the envelope is left in situ, it becomes part of the implant. Alternatively, the envelope can be a resorbable material that is a temporary component of the implant. Removal of the envelope enables contact between the tissues and the wound mandrel filaments. The mandrel can be removed or left as a component of an implant.

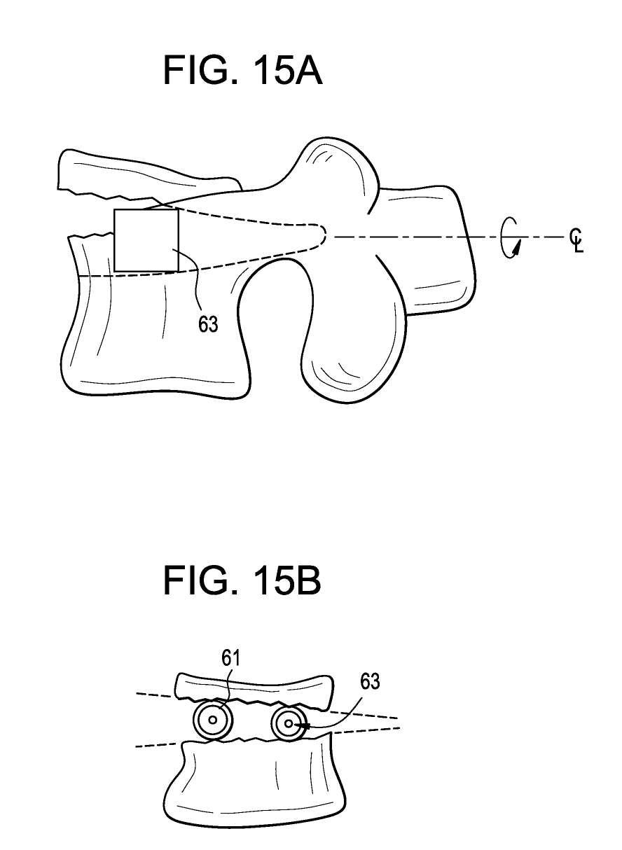

Spools of different sizes placed bilaterally could create a wedge deformity in the vertebral body that enables a scoliotic correction. Now referring to FIGS. 15a-b, there is provided a side and front views of a pair of different-sized spools 61, 63 of the present invention inserted into a vertebral body so as to provide scoliotic correction. Such bilaterally placed spools of disproportionate size could be used as vertebral body augmentation devices, cartilaginous growth plate distractors, or interbody spacers in order to achieve a scoliotic correction. The growth plate distractors (mechanical devices used to mechanically modulate cartilage/bone growth proximal to the growth plate) may be inside the vertebral body or used as external devices attached to growth plate “staples” that use the spool material to promote or constrain growth of the growth plate by imparting contractile or tensile forces on the staple tines.

Soft tissue bulking can also be accomplished by the creation of a spool. Such an implant or distraction device would be useful in genitourinary procedures (sphincter muscle bulking) or various aesthetic procedures (such as breast/calf augmentation).

The method of achieving soft tissue augmentation using a spool has not been described in the literature.

A mandrel is inserted into a tissue space and filament material is wound onto the mandrel. The action of winding the mandrel with filament can effectively rasp or remove local tissues. The action of winding the mandrel with filament can displace local tissues as a tamp. If left in situ, the assembly can act as a soft tissue augmentation material. If unwound and removed, the assembly will leave a tamped and/or excised soft tissue space. If the filament contained a dehydrated material (biocompatible hydrogel), local fluids would hydrate the filament and swell the assembly. The final swollen assembly could act as a tamp or augmentation implant. The mandrel could act as a foramen with filament material acting to stabilize the device. The mandrel could be a percutaneous device, transcutaneous device, or completely implanted device. The assembly could be a temporary or permanent implant.

As the baby boomer population ages, it is expected that this population will experience an increasing amount of osteoporosis. Since it is expected that increased osteoporosis increases the prevalence of bone screw pullout, there is an increasing need for technologies that will prevent bone screw pullout.

Therefore, in some embodiments, the spool of the present invention is used to stabilize a bone screw, preferably a pedicle screw.

In some embodiments, the spool is situated distal of the bone screw, so as not to interfere with the purchase of the bone screw in the osteoporotic bone.

In some embodiments, the filament is fed to the mandrel through a channel on the outer surface of the bone screw. This channel protects the filament during bone screw insertion.

The mandrel may be disposed on a rod that passes through a cannulated screw in a manner similar to an obdurator. The distal end of the mandrel has a lip thereon so as to form an enlarged distal head thereon having a substantially similar diameter to the bore in the screw. The filament is joined to the mandrel on the underside of the lip, and so is protected from high stresses during bone screw insertion. The filament is fed onto the mandrel through a stationary eyelet disposed on the screw annulus.

In some embodiments, the filament comprises an acrylic material, such as a methacrylate such as PMMA. Making the filament out of an acrylic such as PMMA is advantageous because PMMA has been well characterized in the context of treating vertebral body compression fractures.

In some embodiments using an acrylic filament, the wound acrylic filament is then sintered to create intra-filament bonds and thereby enhance the stability of the spool. Sintering the acrylic filament is also advantageous because it may reduce or eliminate the need to add a slurry of conventional PMMA cement around the spool.

Without wishing to be tied to a theory, eliminating the need for a slurry of conventional PMMA cement (via sintering) may provide the clinician with a number of important advantages. First, eliminating the slurry of conventional PMMA cement eliminates the patient and clinician exposure to the slurry's toxic reactants such as the MMA monomer and dmpT. Second, eliminating the slurry reduces the pressure produced within the vertebral body during slurry delivery from an injector gun, and so eliminates embolisms. Eliminating cement extrusion from the vertebral body, decreasing or eliminating the high exotherm associated with PMMA curing, and eliminating bone cement syndrome are also benefits to a sintering system. Third, eliminating the slurry of conventional PMMA cement eliminates the concern over losing control over the slurry's deposition within the vertebral body, and so eliminates the need for the surgeon to use X-rays to monitor the movement of the slurry. The advantages of eliminating the need for radiographic control not only eliminates surgeon exposure to harmful X-rays, it also eliminates the need to have a radioopaque agent (such as barium sulfate) in the PMMA. Eliminating the barium sulfate from the PMMA filament will likely enhance the strength of the sintered PMMA filament. Therefore, in some embodiments, the filament consists essentially of the acylic material. In sum, there appear to be several advantages to eliminating the use of a conventional PMMA slurry from the present invention.

In general, PMMA typically has a glass transition temperature (Tg) of at least about 90-100° C. Producing such high temperatures in the patient in order to sinter the wound filament may compromise the patient's living tissue. Therefore, in preferred sintered embodiments, additional steps are taken in order to lower the sintering temperatures of the wound acrylic filament. The goal is to control the peak temperature of local tissue exposure. In some sintering embodiments, heat can be added to the mandrel or spool core, then extracted from the device before local tissues experienced significantly increased temperatures.

In some such sintered PMMA embodiments, the outer surface of the PMMA filament is pre-oxidized. It has been reported by Tsao, Lab Chip, 2007, 7, 499-505, that pre-oxidized PMMA can be sintered at temperatures in the range of about 70-90° C. and thereby produce bonded PMMA bodies having a strength essentially equivalent to a pure PMMA body that has been sintered 20° C. above its glass transition temperature (Tg). In some embodiments, the filament consists essentially of oxidized PMMA.

In some embodiments, the PMMA has a Tg of no more than 85° C. In some embodiments thereof, the PMMA is CMW1, available from CMW, Blackpool, UK. It has been reported that the CMW cements have a Tg of about 71-72° C.

In some embodiments, the PMMA filament has a ribbon shape. In some embodiments, the PMMA filament is tightly wound upon the mandrel. This increases the density of the packing, leading to greater filament-to-filament contact points within the winding, and to enhanced bonding within the sintered body.

Therefore, in some embodiments, the filament comprises oxidized PMMA in a ribbon shape. This embodiment has the advantage of producing a very low porosity (high density) packing which can then be sintered to produce a very low porosity (high density) sintered PMMA body.

Other methods of producing a high density packing may also be employed in accordance with the present invention. For example, once the filament has been wound around the mandrel, a slurry of fine PMMA particles may be introduced into the winding. The PMMA particles in the slurry will deposit within the pores of the winding. Subsequent sintering will produce a low porosity PMMA body.

In one preferred embodiment, heat is delivered to the wound PMMA filament through a lavage treatment. In another, heat is delivered by transmitting high intensity light through the PMMA filament. In another, heat is transmitted by inserting a resistance wire into the bore of the mandrel and applying a voltage thereto. In another, heat is generated by radio frequency energy transducers located within the filaments to create local heat depots.

In a preferred heat treatment, the PMMA filament is heated to a temperature of between 50° C. and about 100° C., and such temperature is sufficient to achieve sintering. Preferably, the PMMA filament is heated to a temperature of between 70° C. and 90° C., and such temperature is sufficient to achieve sintering.

Pressure may also be delivered to the PMMA winding during sintering. In one embodiment, the PMMA filament is tightly wound upon the mandrel. The tight winding produces high compaction forces in the filament, thereby enhancing the sintering efficacy. In another embodiment, high pressure fluid is introduced around the wound filament. This has the effect of enhancing the intra-filament bonding of the PMMA, and so strengthens the spool. Alternatively, the internal windings of the spool could be displaced outwards to create internal pressure from within the spool towards the periphery of the spool that can help sinter the PMMA. The mandrel could expand to create this internal pressure.

In one preferred embodiment, heat and pressure are delivered to the wound PMMA filament through a lavage treatment.

In some embodiments, the ribbon is introduced upon a cannula whose bore a) has a ribbon cross-section and b) has a distal curve so that it opens so as to face the mandrel.

Because it is believed that micromotion is a pain generator in vertebral body compression fractures, it is desired to establish a way to lock the spool in place in the vertebral body.

In some embodiments, the clinician may form a tapered spool having a perimeter that substantially defines a Morse taper, and then move the tapered spool along its longitudinal axis to seat the implant. Moving the Morse taper spool along the longitudinal axis has the effect of tamping the bone and taper locking the spool, thereby forming a lock-and-key fit between fracture planes.

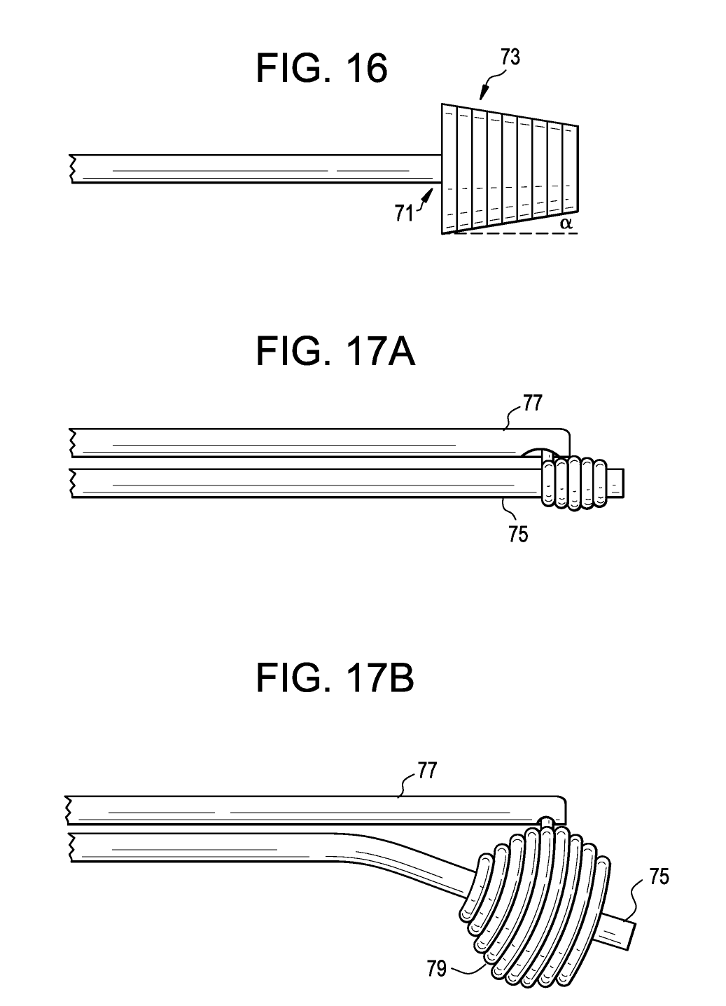

In some embodiments thereof, and now referring to FIG. 16, the filament is wound in a predetermined frustoconical shape upon the mandrel 71 so that the resultant winding 73 has a frustoconical angle α of less than about 25 degrees (and preferably less than 10 degrees). When the spool has such a low angled winding, it is believed that, upon subsequent axial advance of the spool into the vertebral body, the winding forms a taper lock with the cancellous bone of the vertebral body.

It is believed that causing the spool to be taper-locked in the vertebral body is a very advantageous aspect of the present invention. The production of the taper lock means that the spool is locked in place, and so will not migrate in the vertebral body. Accordingly, the spool will not be predisposed to micromotion. Because micromotion is believed to be a principal source of pain in a patient having a vertebral body compression fracture, and has been cited as a principal cause of pain in kyphoplasty procedures, the taper lock feature of the present invention is a very valuable one.

In addition, the enhanced stability provided by the taper lock may also obviate the need to use a flowable bone cement as a grouting agent for the spool. It is believed that eliminating the use of the flowable bone cement will be highly desirable, as issues such as extravasation and reactant toxicity are thereby eliminated.

In some embodiments, the spool is stabilized in the vertebral body by a combination of the taper lock and the sintering of the filament within the winding. In some embodiments, the winding is sintered before axial advance of the spool. This insures that the winding acts as an integral body during the taper locking of the spool. In some embodiments, the sintered winding is re-sintered after axial advance of the spool. The re-sintering step causes the filament to adhere to the peripheral bone, thereby decreasing the possibility of micromotion even more.

The concept of interference fitting an implant in bone is known in the art. For example, bone dowels operate on the principle of an interference fit. Since the simple axial advance of the furstoconical winding into the bone should cause the spool to become taper locked therein, there is provided a simple way to stabilize the implant and reduce pain as well.

In general, it is believed that the lower the angle of the frustocone, the greater the mechanical lock of the spool in the vertebral body. Therefore, in some embodiments, the taper angle α of the frustocone is less than 18 degrees, preferably less than 10 degrees, more preferably less than 6 degrees.

In the taper-locked embodiments, the filament may be chosen so that the winding can more closely approximate a frustocone. Thus, in some of these embodiments, a relatively small diameter filament (e.g., a less than one mm diameter) may be chosen. In other taper-locked embodiments, the filament has a ribbon shape.

It is anticipated that, after a short (e.g., 3 mm) axial advance of the spool into the vertebral body, there may be a small space created directly adjacent the posterior end of the winding. This space can be filled with the final windings of the filament in the vertebral body.

Because access to the vertebral body through the pedicle generally places the spool in a substantially off-center location, many vertebroplasty procedures are performed bilaterally. Typically, the clinician undertakes a pair of procedures, with one procedure carried out through each respective pedicle. However, because vertebroplasty procedures are considered to be expensive, there is a desire to perform an entire vertebroplasty procedure unilaterally (i.e., through a single pedicle).

Therefore, it is an object of the present invention to provide a spool that can treat an entire vertebral body through a single pedicle.

In accordance with this object, in some embodiments, the apparatus of the present invention comprises a relatively stiff guide and a relatively flexible mandrel. When these two components are placed alongside each other in the vertebral body, and as the filament material accumulates upon the mandrel, the winding will soon begin to butt up against the guide. Because the stiff guide will not cede ground to the encroaching mandrel, subsequent accumulation of filament upon the flexible mandrel will result in a deflection of the mandrel and the winding away from the guide. If the guide is placed at a location lateral to the mandrel, the deflection of the mandrel will be towards the centerline of the vertebral body.

It is believed that such a deflection of the mandrel and winding may be sufficiently large so as to effectively place the spool substantially close to the midline of the vertebral body. When the spool is placed substantially along the midline, its location is sufficiently centered so that only a single spool needs to be implanted in a vertebral body.

Now referring to FIGS. 17a and b, there is provided an example of the deflecting mandrel. In FIG. 17a, the mandrel 75 and guide 77 are located directly adjacent one another. Thereafter, initial growth of the winding upon the mandrel causes the mandrel to slightly deflect away from the stiff guide. In FIG. 17b, more significant filament deposition causes even greater growth of the winding 79, and so even greater deflection of the mandrel towards the center of the vertebral body.

In some embodiments, the deflection of the spool is carried out by an articulating mandrel. This feature may include the use of a universal joint or series of universal joints. The articulation means can include machined springs that impart very high torsional forces while preserving their diameter and length characteristics. The articulation means can alternatively include braided wire or tubing in order to preserve a cannulation within the flexible (articulating) portion of the device. Alternately, the mandrel could be attached to the balance of the longitudinal rod through an articulating gear system.

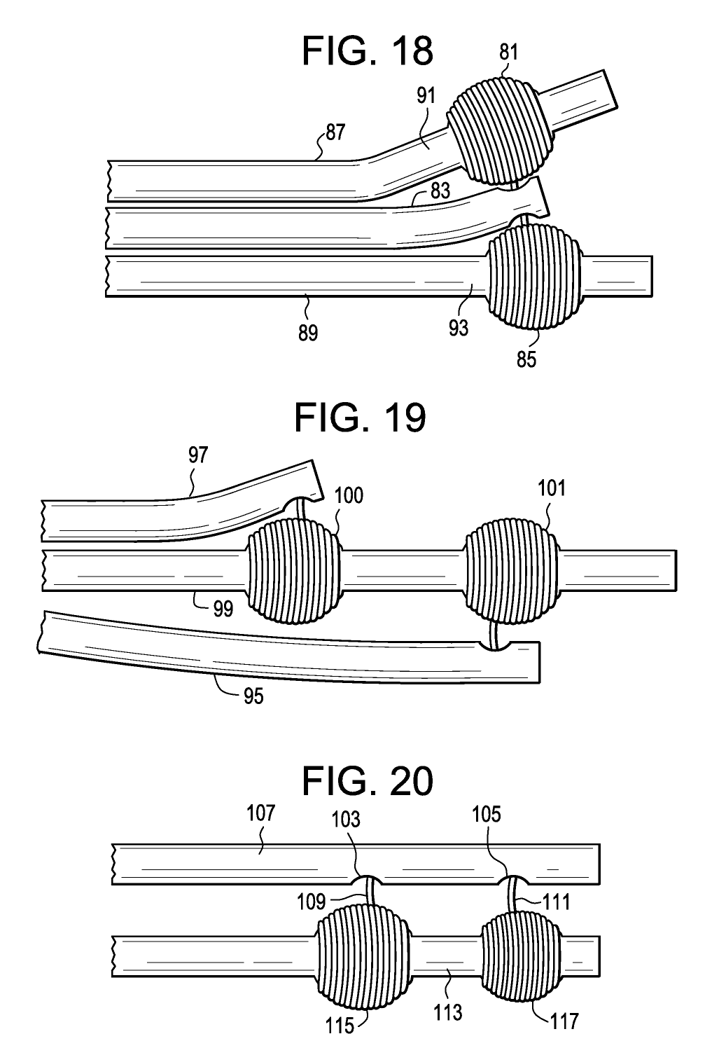

In some embodiments, the apparatus of the present invention comprises first and second spools. In one embodiment thereof, the apparatus comprises first and second rods, with first and second mandrels at the distal ends of the rods. Typically, as in FIG. 18, the spools 81 and 85 are provided in a side-by side relationship. The delivery of two side-by side spools through the same pedicle allows the clinician to accomplish fracture reduction and height restoration along a wider plane. Preferably, a first spool 81 is set in a medial location and is flexible, while a second spool 85 is set in a lateral location and is rigid. In this condition, the medial spool will deflect inwards as its winding grows. Its ultimate location should be even more medial, as it is deflected inward by the medial portion of the lateral spool. In this embodiment, the filament guide 83 is located between the rods 87 and 89 and provides filament to each of the mandrels 91 and 93

In some embodiments, the two spools may receive independent filaments from the same guide. In these embodiments, the guide preferably has a pair of diametrically opposing openings through which the filaments are delivered (as in FIG. 18).

In some embodiments, and now referring to FIG. 19, the apparatus of the present invention comprises first 95 and second 97 filament guides. The delivery of two guides through the same pedicle allows two filaments to be deposited upon the same mandrel 99, and therefore allows the creation of two different windings 100, 101 upon the same mandrel. In some embodiments, the two windings allow the spool to form an overall dog-bone shape (as in FIG. 19). Without wishing to be tied to a theory, it is believed that the dogbone shape is advantageous because each head (winding) of the dogbone help resists axial motion along the longitudinal axis of the dogbone. In some embodiments, the two filaments consist of different materials or properties thereby enabling different geometries or biological properties to be achieved simultaneously.

In some embodiments, and now referring to FIG. 20, the apparatus of the present invention comprises first 103 and second distal openings 105 upon the same filament guide 107. In this embodiment, first 109 and second 111 filaments are fed into the guide through a proximal opening and exit through respective first and second distal openings. The delivery of two filaments upon the same mandrel 113 allows the creation of two different windings 115, 117 upon the same mandrel. In some embodiments, the two windings allow the spool to form an overall dog-bone shape (as above). In some embodiments, the two distal openings are located in substantially the same radial position on the guide cross-section, and preferably face the mandrel (as in FIG. 20).

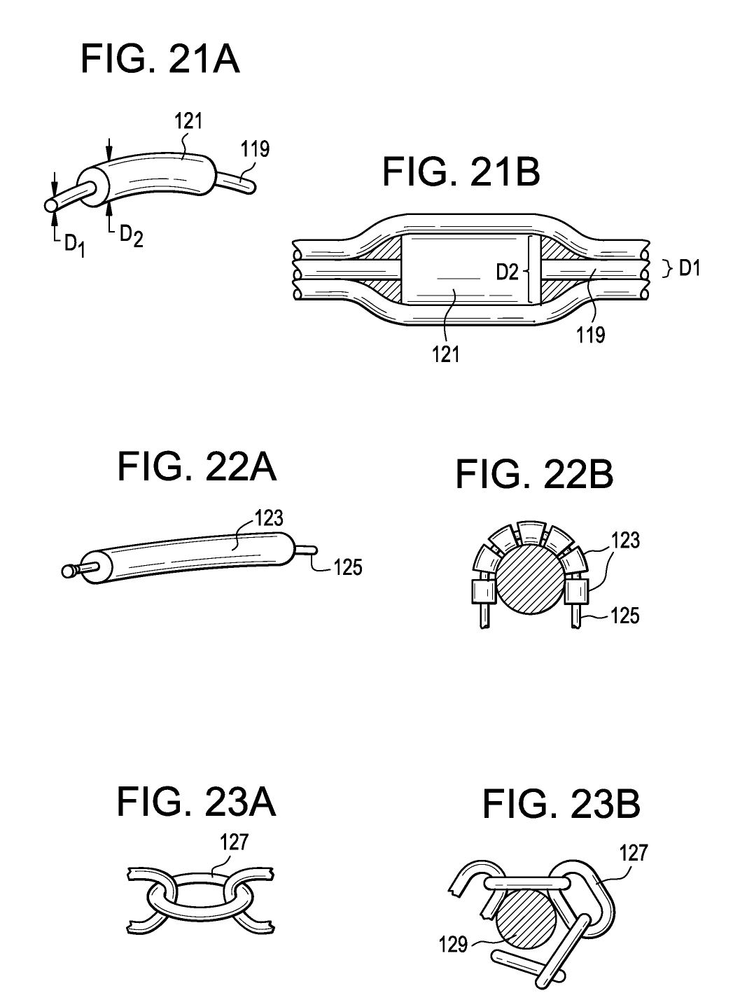

In some embodiments, the filament contains discontinuities. The discontinuities can be designed to reduce the packing efficiency of the winding and thereby impart a predetermined porosity into the winding. For example, as shown in FIG. 21a, there is provided a filament 119 having a discontinuity 121 having a cylindrical shape. This filament can be fabricated by overmolding a cylindrical shape onto a standard filament. As shown in FIG. MF21b, the pore size produced by a winding of this filament can be manipulated by varying the relative diameters of the standard filament (D1) and the overmolded cylindrical discontinuity (D2). In this case, the pore size of the winding will be determined by the equation pore size=½ (D2−D1).

In some embodiments, and now referring to FIGS. 22a and b, the discontinuities 123 on the standard filament 125 are closely spaced and have a diameter far greater than that of the standard filament. In such a case, the filament will substantially take on the shape of a continuous, curving discontinuity. Such a shape is set out in FIG. 22b. The advantage of this shape is that a spool having the desired height can be fabricated with very few turns of the filament, thereby reducing the time required to produce the desired height.

In some embodiments, and now referring to FIGS. 23a and b the filament is a chain link having a plurality of linkages 127. This chain link can wrap around the mandrel 129 easily without breaking. It also provides a measure of porosity in the winding.

In some embodiments of the present invention, the longitudinal rod is rotated by a device drive, such as a drill. However, there is typically substantial radiopacity associated with a drill, which significantly obstructs imaging. Therefore, in some embodiments, the longitudinal rod of the present invention includes an angular offset gear that allows the drill to provide drive power to the rod without obstructing c-arm imaging. As the offset gear mechanism may also create imaging artifacts that need to be minimized, it is preferred that radiolucent materials be used for the offset gear. In some embodiments thereof, PEEK or carbon fiber is the material of construction for the shafts and gears of the angular offset. In some embodiments, the drive mechanism includes quick detachment means for better visualization.

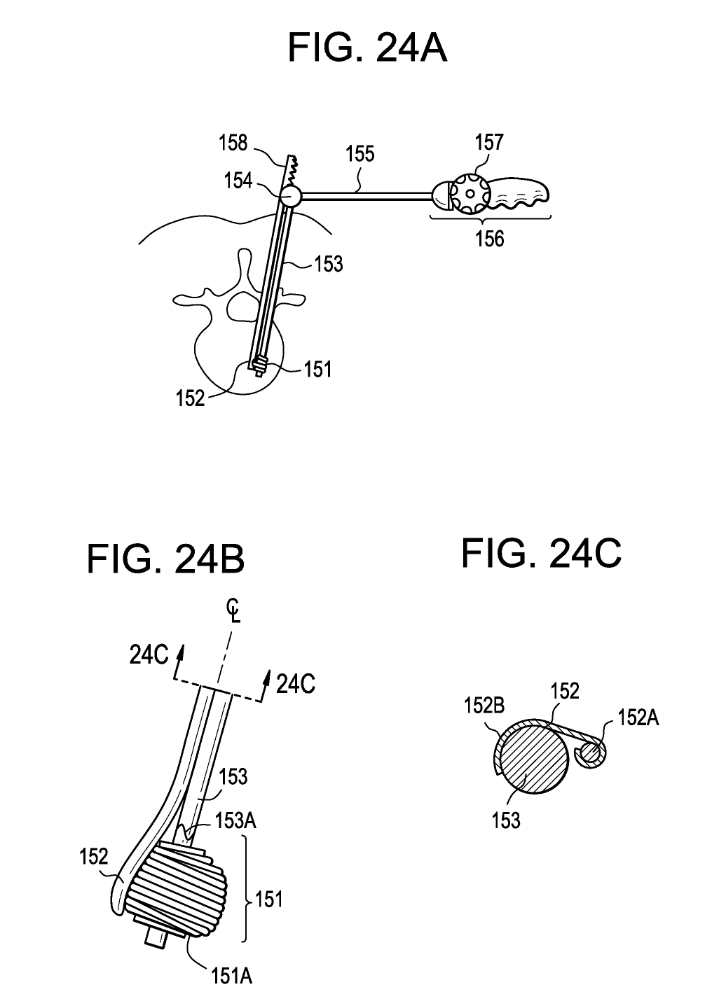

An example of an offset gear will now be described. Now referring to FIGS. 24a-c, there is provided an offset device of the present invention, comprising:
a) a longitudinal rod comprising i) a distal rod 153 comprising a distal mandrel having a detachment means 153A and a proximal depth adjustment rack 158 comprising a plurality of teeth 159, ii) a proximal rod 155 comprising a distal angled torque transmitter 154 and a proximal handle 156 having a depth adjustment knob 157,
wherein the distal angled torque transmitter engages the teeth of the distal angled torque transmitter,
b) a filament guide cannula 152 having a proximal end and a distal end, and
c) a filament 151A exiting the distal end of the filament guide cannula and wound around the mandrel to create a spool 151.

In use, handle 156 is powered to rotate rods 153 and 155. The depth of the distal end of the filament guide cannula 152 in the vertebral body is controlled by adjusting the depth adjustment knob 157. The engagement of the distal angled torque transmitter in the teeth of the distal angled torque transmitter provide an angled offset in the device, thereby allowing the clinician to avoid obstructing the C-arm.

Now referring to FIG. 24, there is provided a distal cross-section of the offset device, describing how the filament guide cannula is sufficiently flexible to wrap around the distal portion 153 of the rod and provide a channel 152A for feeding the filament to the mandrel. Thus, in some embodiments, the filament guide cannula 152 has a) a flexible portion providing a C-shaped cross-section 152 B and b) a channel portion 152 A for delivering the filament.

In these preferred embodiments, the filament guide is designed with a certain amount of flexibility similar to a standard tape measure, wherein the guide is rigid in one plane but flexible in a transverse plane, with adequate longitudinal stiffness in the transverse plane. This feature allows the proximal portion of the filament guide to wrap around the longitudinal rod while its distal portion can flex outwards to accommodate an increasing spool diameter (as shown in FIG. 24b).

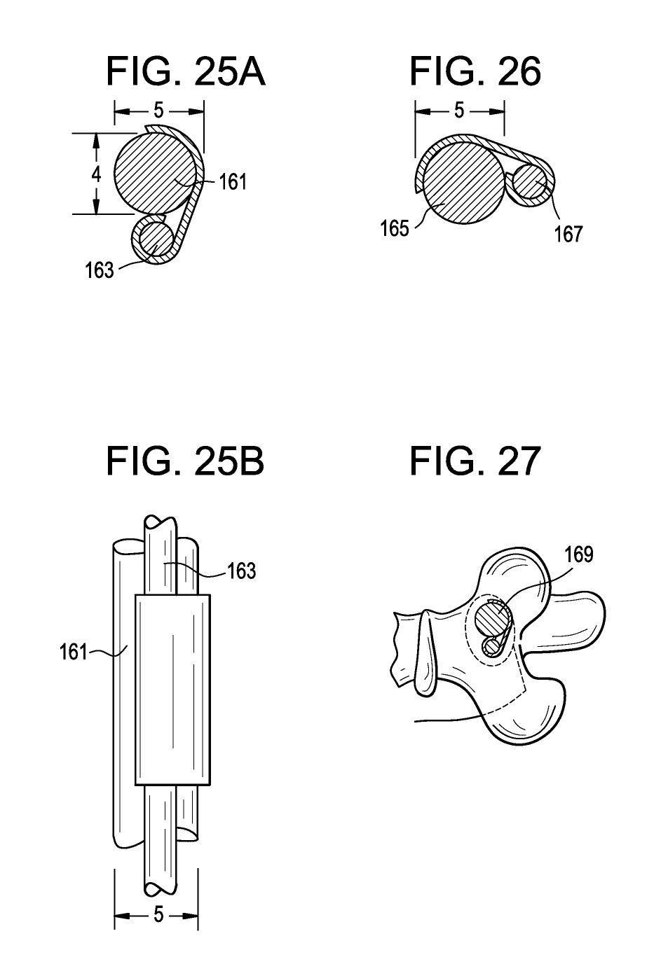

Now referring to FIGS. 25 a and b, in one preferred embodiment, in the pedicular region, the diameter of the longitudinal rod 161 is about 4 mm, and the diameter of the filament 163 is about 1.5 mm. Given these dimensions, the cross-section of the device in the pedicle should be about 7 mm×5 mm.

Now referring to FIG. 26, in another preferred embodiment, in the pedicular region, the diameter of the longitudinal rod 165 is about 3 mm, and the diameter of the filament 167 is about 1 mm. Given these dimensions, the cross-section of the device in the pedicle should be about 5 mm×4 mm.

Thus, filament diameter plays an important role in the pedicular cross-section of the device. It is also determinative of the number of turns needed to accumulate a desired amount of material on the spool, as well as winding porosity and procedure time.

Now referring to FIG. 27, there is provided a cross-section of a device of the present invention traversing a pedicle, wherein the pedicle is represented by an ovoid dotted line. In this embodiment, wherein the long dimension of the pedicle is about 8 mm, the diameter of the longitudinal rod is about 3 mm, and the diameter of the filament is about 1 mm. It is shown that the ovoid cross-section of the device 169 in the pedicular region substantially parallels the larger ovoid shape of the pedicle cross-section. Thus, the substantial correspondence of these shapes means that the contemplated device can be conveniently designed to easily fit within a typical pedicle.

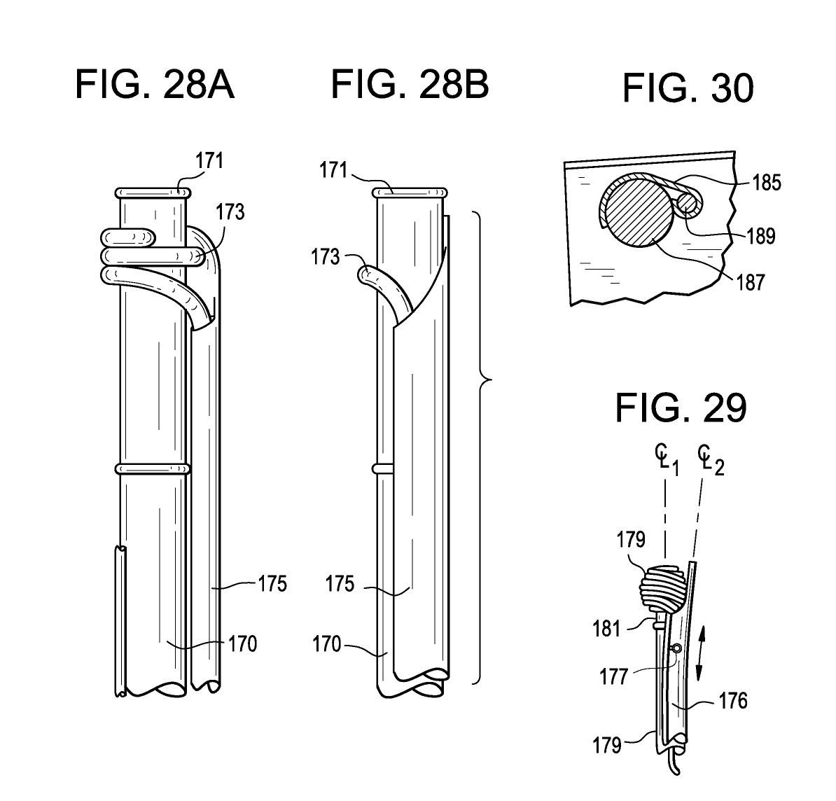

Now referring to FIGS. 28 1a and b, there is provided bottom and side views of the distal end portion of a device of the present invention, including a longitudinal rod 170 comprising a mandrel 171, a filament 173, and a filament guide 175. Whereas the bottom view image shows several filament windings on the mandrel, the side view does not. To accommodate these windings, the filament guide cannula will have to deflect away from the spool.

Now referring to FIG. 29, in order to accommodate the spool growth, the centerline CL2 of the filament cannula 176 will need to deflect away from the mandrel centerline CL 1. Accumulated filament winding 179 displaces the cannula tip. A recess 177, shown at the bend point on the cannula, helps the cannula achieve the desired deflection. This filament cannula enables jam-free filament deployment at various locations on the mandrel 181.

Now referring to FIG. 30, the cannula 185 acts as a spring clip on the longitudinal rod 187, while delivering the filament 189. This enables sliding longitudinal motion while approximating parts.

These various filament and mandrel embodiments can be designed to enable jam-free filament deployment and filament recall (removal of the filament from the mandrel, if necessary). It may be necessary to spool filament, then unspool all or some of the accumulated filament to achieve the desired effect.

In one embodiment, a depth gauge is used to measure the distance into the patient's body. This can be accomplished by establishing a datum plane on the patient's skin and/or vertebral body. Depth measurements can be made pertaining to the device's depth from the epidermis and penetration into the vertebral body.

In one embodiment in which the spool is locked in place, the mandrel is set at a fixed depth with respect to the posterior cortical edge of vertebral body. This setting of the depth is expected to provide both a safety feature as well as a clinical outcome feature. To lock the depth of the mandrel within the vertebral body, the surface of the vertebral body or patient's skin can be established as a datum plane. The cortical bone at the device's entry point into the vertebral body is a preferred locus for establishing a datum plane. This may be achieved by inserting the assembly through an introducing cannula whose distal end terminates at the posterior cortical shell of the targeted pedicle. The mandrel can be set at a depth fixed within the introducing cannula, and the introducing cannula can be fixed at the cortical shell upon device insertion. In this embodiment, the mandrel is free to rotate, but is fixed with respect to axial location. This location fixation can be achieved by providing a shoulder on the mandrel (or longitudinal rod) that interfaces with a recess on the introducing cannula. The cannula is able to slide until the desired depth is established, at which time the sliding engagement is fixed so as to stop all sliding motion. The introducing cannula can be buttressed against the cortical shell using a deep flair or projections that prevent further penetration into the vertebral body.

A similar arrangement can be made with respect to the patient's skin. Simply, instead of using the cortical shell as the datum, the epidermis local to the insertion point is selected. However, this embodiment is less desirable, since the skin is a flexible and compressible structure. As fluids and elastic soft tissue properties change during the procedure, the relative locations of the mandrel and vertebral body could change if the skin is used as the datum plane.

In preferred embodiments, the longitudinal rod's angular trajectory into the pedicle and vertebral body is substantially maintained through the step of spooling. However, because the lever arm provided by the rod is long enough to enable the clinician to core out the trabecular bone simply by moving the device's handle outside of the body, careless or undesired movement might compromise the desired implant placement or create a hazardous clinical situation (such as anterior or lateral breach into the great vessels). Therefore, care must be taken to maintain the initial rod trajectory (even though it may be desirable to allow the implanted spool to reorient during the operation, i.e., the centerline of the spool may be allowed to diverge from the longitudinal rod's centerline). The same mechanism used to fix mandrel depth can be employed or modified to enable maintenance of the mandrel's trajectory. Using a flexible drive shaft to create the mandrel rotation can help to prevent moment loads on the mandrel shaft. Such a flexible torsion shaft could connect directly with the spool, with the mandrel within the patient, at the datum entry, at the skin level, or outside the patient. Since a flexible drive shaft is incapable of imparting substantial moment loads, but very capable of imparting torsional loads, this embodiment may be preferable to fixing the entry angle of a rigid mandrel torsion-shaft.

In some embodiments, the device drive possesses multiple drive speeds that dictate the speed at which filament is deposited. A rapid filament deposition speed might be desired at the beginning of the procedure in which a simple bone tamping is being performed. A slower filament accumulation speed might be desirable for when fine spool shaping and/or deliberate fiber placement is undertaken. In some embodiments, the initial windings of the spool could be deposited by using larger diameter filament (so that deposition proceeds quickly) while the subsequent windings could be constructed from smaller-diameter filament (that enables more control but requires many more mandrel turns).

In some embodiments, the clinician is able to finely control the volume of filament deposited on the mandrel, and thereby insure that filament is not excessively deposited. In these embodiments, the clinician may use direct imaging to observe the spooling and subsequent cement injection operations. However, it may be desirable to be able to measure filament deposition by means other than an x-ray.

Without wishing to be tied to a theory, it is believed that the volume of deposited fiber will likely correlate with the length of fiber drawn onto the mandrel. Although there is no definitive way to measure fiber packing efficiency in situ, it is estimated that, due to packing inefficiencies, in some embodiments, the winding volume will be between about 20% and about 30% greater than the volume of the deposited filament. Therefore, in some embodiments, the volume occupied by the deposited filament may be estimated by a correlation between winding volumes and length of deposited filament. This may provide a convenient way to measure filament deposition by means other than an x-ray.

It may be desirable to use radiographs to estimate the volume of spool within the patient. An image of the spool represents a planar projection of the spool profile. Revolving the projection 180° (or rotating one-half the projection about it's center-line 360° will create the net volume of material being displaced within the vertebral body. The ratio of filament volume to this rotated projection volume represents the solid portion of the spool, whereas the projection only represents the filament volume and porosity within the spool. It may be desirable for surgeons to have porosity estimations during the operation so they can control or monitor spool porosity. A more porous spool may have greater mechanical compliance than a less porous spool. If spool porosity correlates with mechanical compliance of the implant, then surgeons may be able to control (or monitor) the mechanical compliance mismatch between bone and the spool.

In preferred embodiments, the shape of the winding is intraoperatively determined by the clinician. Preferably, this is accomplished by adjusting the relative positions of the mandrel and the distal end opening of the filament guide cannula during filament deposition. Desirably, the filament guide cannula and mandrel are designed to control the ultimate size and shape of the spool. In preliminary experiments, it has been demonstrated that it is possible to shape the spool by altering the placement of the distal end opening of the filament guide cannula relative to the mandrel. In preferred methods of intraoperative shaping of the spool, the clinician moves the cannula along its longitudinal axis (while keeping the mandrel substantially axially fixed). However, in other embodiments, the clinician may move the mandrel relative to the cannula.

It is appreciated by the present inventors that the spooling methodology should strike a suitable balance between speed and accuracy. Whereas is would be undesirable for the clinician to spend significant time repeatedly turning a hand crank, it would also be undesirable for an overpowered drill to instantly produce an over-sized winding in the vertebral body. It is believed that the spooling speed must enable controllable spooling of the implant while maintaining a reasonable operative time.

In some embodiments, there is provided an assembly comprising a power drill attached to a hand-operated screwdriver. Preferably, the screwdriver is an “in-line” screwdriver that can also be rotated by hand. At any time during the spooling procedure, the physician using such a device may convert the spooling from a power drill-driven spooling process to a hand drill-driven process in order to obtain a tactile feel for the spool. In some embodiments, the assembly can include either planetary or transmission gear systems that can enable both the desired gear ratio and the desired tactile input.

In some embodiments, the handle of the longitudinal rod contains a power driver that can be mated to a hand-crank. When the power driver is engaged, the exterior of the handle is uninvolved except for driver control features. When the hand-crank is engaged, a clutch mechanism engages with the power driver and the torque rod is driven by a more direct tactile input.

Desirably, the spool has an elastic modulus between about ⅓ that of cancellous bone and three times that of cancellous bone. However, in some embodiments, the spool has a relatively lower elastic modulus. In other embodiments, the spool has a relatively higher elastic modulus.

In general, it is believed that the filament will have a higher intrinsic modulus than the surrounding cancellous bone and so it is believed that the spool would have a higher modulus than the surrounding cancellous bone. However, in some embodiments, it may be desirable to provide a bladder around the mandrel that can be filled or inflated with low modulus materials in order to modulate the mechanical properties of the spool. Implant compliance can be designed by material properties, spool geometry, filament geometry, and spool placement.

If the spool is then overfilled with flowable cement to stabilize the spool device, then mechanical compliance may be an issue, as current bone cements are relatively stiff. Thus, in some embodiments, elastic cements may be desirable.

In some embodiment, a calcium salt bone void filler is used as the overfill material in order to provoke remodeling around the spool device.

In some embodiments, the implantable filament is selected from the group consisting of PP hernia mesh filament, PET/Dacron, sternotomy wire, S of wire, CoCr metal suture oro circlage wire, ultra-high molecular weight polyethylene, and PTFE. In some embodiments, the filament comprises a metal that provides radio-opacity and flexibility to the final winding.

In embodiments in which the spool is an implant, it is desirable to provide a facile method of disengagement of the spool from the longitudinal rod. In preferred embodiments, the proximal end of the spool can be detached from the longitudinal rod through the use of splines, debonded adhesives, heat, or fracture. The spool can be detached from the longitudinal rod by an interface, sheath, heat, chemical release, PMMA injectate dissolution, unspooling action after spool installation (reversing torque rod direction could unscrew the rod from the mandrel or unspool the initial spool windings to enable device removal).

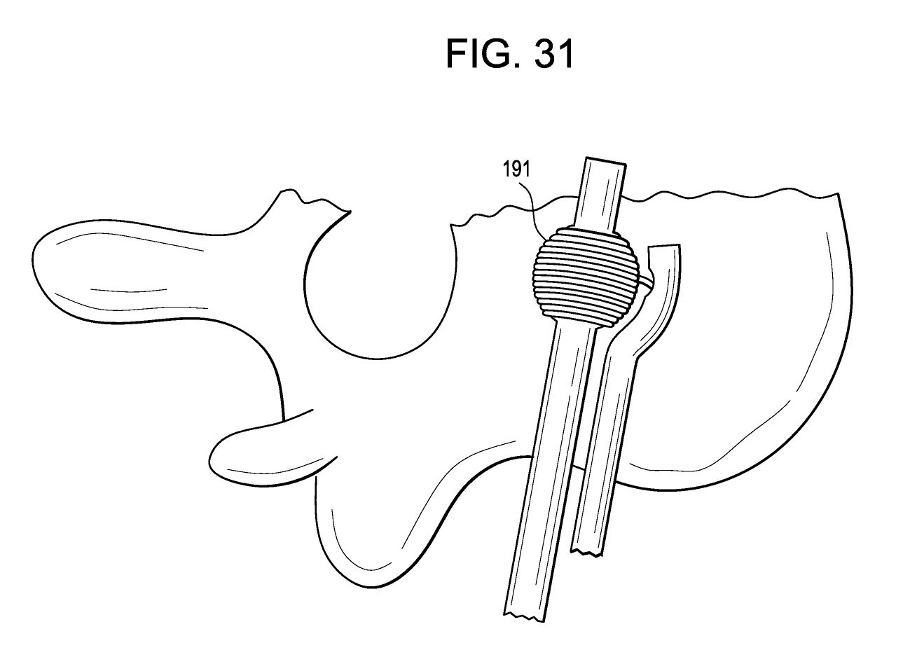

Now referring to FIG. 31, because the spooling mechanism of the present invention generally creates a radial symmetry in the final spool, it may be desirable to enter the vertebral body through an extrapedicular approach (that results in a mandrel having a more medial location) to ensure that the growing spool 191 does not encroach on the lateral aspect of the vertebral body. An extrapedicular approach (as shown in FIG. 31) may also give the clinician room to grow the spool diameter.

We claim:

1. A vertebral body distraction device, comprising;
a) a mandrel;
b) a filament attached to the mandrel,
wherein the filament is wound around the mandrel to produce a winding,
wherein the filament comprises an acrylic,
wherein the acrylic filament has a glass transition temperature of less than 80° C.

2. The device of claim 1 wherein the filament has a surface comprising oxidized PMMA.

3. The device of claim 1 wherein the filament comprises an additive that lowers the glass transition temperature of acrylic.

4. The device of claim 1 wherein the winding comprises a pore space, and wherein the device further comprises:
c) acrylic cement located within the pore space of the winding.

5. The device of claim 1 wherein the winding is sintered.

6. The device of claim 1 wherein the winding comprises an outer periphery, the device further comprising:
c) acrylic cement located about the outer periphery of the winding.

7. The device of claim 1 wherein mandrel comprises an acrylic.

8. The device of claim 1 wherein the mandrel comprises a bore having a distal opening than opens upon the winding.

9. The device of claim 8 further comprising:
c) an acrylic cement located within the bore of the mandrel.

10. The device of claim 1 wherein the acrylic filament comprises discontinuities.

11. The device of claim 1 wherein the acrylic comprises a methacrylate.

12. The device of claim 11 wherein the methacylate comprises PMMA.
